(19) United States
(12) Patent Application Publication
(10) Pub. No.: US 2025/0288543 A1
(43) Pub. Date: Sep. 18, 2025

(54) METHODS OF ENHANCING BENEFICIAL SHORT-CHAIN AND MEDIUM-CHAIN FATTY ACIDS PRODUCTION BY GUT MICROBIOME

(71) Applicants: Anca DELGADO, Tempe, AZ (US); Rosa KRAJMALNIK-BROWN, Chandler, AZ (US); Dorsa DAEIZADEH, Tempe, AZ (US)

(72) Inventors: Anca DELGADO, Tempe, AZ (US); Rosa KRAJMALNIK-BROWN, Chandler, AZ (US); Dorsa DAEIZADEH, Tempe, AZ (US)

(73) Assignee: Arizona Board of Regents on Behalf of Arizona State University, Scottsdale, AZ (US)

(21) Appl. No.: 19/078,134

(22) Filed: Mar. 12, 2025

Related U.S. Application Data
(60) Provisional application No. 63/564,485, filed on Mar. 12, 2024.

(51) Int. Cl. A61K 31/19; A61K 9/00; A61K 31/045

(52) U.S. Cl. CPC A61K 31/19; A61K 9/0031; A61K 31/045

(57) ABSTRACT

Disclosed herein are methods and compositions related to increasing beneficial short-chain and medium-chain fatty acids production by gut microbiome. The beneficial short-chain and medium-chain fatty acids include, for example, butyrate and coproate. The compositions comprise a substrate for chain elongation by gut microbiome selected from ethanol, lactate, or a combination thereof. In some aspects, the compositions are formulation for colonic delivery.

### RELATED APPLICATIONS

This application claims priority to and the benefit of U.S. provisional patent application 63/564,485, filed Mar. 12, 2024 titled “Methods of Enhancing Beneficial Short-Chain and Medium-Chain Fatty Acids Production by Gut Microbiome,” the entirety of the disclosure of which is hereby incorporated by this reference.

### STATEMENT REGARDING FEDERALLY SPONSORED RESEARCH OR DEVELOPMENT

This invention was made with government support under 2221805 awarded by the National Science Foundation. The government has certain rights in the invention.

### TECHNICAL FIELD

This document relates to the identification of ideal conditions for chain elongation by gut microbiome that leads to enhanced production of short-chain and medium-chain fatty acids.

### BACKGROUND

Therapeutic applications of MCFAs include but are not limited to:
1. Metabolic Disorders: MCFAs are used to treat and potentially prevent metabolic diseases such as diabetes, obesity, and hypertriglyceridemia by promoting lipid metabolism and weight loss.
2. Neurodegenerative Diseases: MCFAs show promise in treating neurodegenerative diseases like Alzheimer's disease, Parkinson's disease, and ALS (amyotrophic lateral sclerosis), by promoting mitochondrial biogenesis, stimulating autophagy, and improving motor symptoms.
3. Cancer: Capric, caprylic, and caproic acids exhibit anticancer effects by modulating cell cycle genes and promoting apoptosis, particularly in colon cancer.
4. Inflammatory Bowel Disease (IBD): MCFAs, through their anti-inflammatory properties and ability to regulate the intestinal epithelial barrier, could alleviate symptoms of IBD, including ulcerative colitis (UC) and Crohn's disease (CD).
5. Infections: The antibacterial properties of MCFAs against a variety of pathogens offer an alternative to antibiotic use in animals and potentially humans.
6. Cardiovascular Health: MCFAs may benefit cardiovascular health by reversing steatosis and cardiac dysfunction.

The therapeutic applications of Short-Chain Fatty Acids (SCFAs) include but are not limited to:
1. Obesity and Metabolic Health: SCFAs, particularly butyrate, play a role in obesity and energy metabolism by regulating appetite and improving insulin sensitivity.
2. Cardiovascular Disease: SCFAs can protect the cardiovascular system by decreasing atherosclerosis and modulating cholesterol absorption.
3. Inflammatory and Autoimmune Diseases: SCFAs have anti-inflammatory and immunoregulatory effects that could benefit conditions like inflammatory bowel disease, diabetes, and allergies.
4. Neurodegenerative and Neuropsychiatric Diseases: The neuroprotective effects of SCFAs could offer therapeutic benefits for diseases like Alzheimer's disease, Parkinson's disease, and other neuropsychiatric conditions.

Butyrate (C4), a volatile short-chain fatty acid (SCFA), provides numerous health benefits, including food for gut colonocytes, enhanced cognitive function, and antimicrobial and anti-inflammatory effects. Caproate (C6), a volatile medium-chain fatty acids (MCFA), similarly affects gut microbiota, offering therapeutic potential for metabolic and neurological disorders, and has demonstrated anti-cancer and anti-inflammatory properties. The known pathways for production of butyrate by the colonic microbiome involve fermentation of either carbohydrates, organic acids, or amino acids. Caproate is not a product of conventional carbohydrate fermentation, and its production has not been demonstrated microbially in the colon. Diseases that benefit from increased butyrate and caproate production include, but are limited to diabetes, obesity, Alzheimer's disease, Parkinson's disease, Amyotrophic Lateral Sclerosis (ALS), inflammatory bowel diseases (ulcerative colitis, UC, and Crohn's disease, CD), cancer (particularly colon cancer), and cardiomyopathies.

### SUMMARY

Disclosed herein are compositions comprising a substrate for chain elongation by gut microbiome, wherein the composition is formulated for colonic delivery. The substrate for chain elongation is selected from ethanol, lactate, or a combination thereof. In some aspects, the composition further comprises acetate or butyrate. In some implementations, the composition comprises a colonic delivery system that encapsulates the at least one substrate for chain elongation.

In some aspects, the colonic delivery system comprises a plurality of polymers, wherein at least one polymer of the plurality of polymers prevent penetration of gastric fluid into the colonic delivery system and the plurality of polymers further comprises a pH-independent erodible polymer that delays the release of the at least one substrate for chain elongation for at least four hours after gastric emptying. In certain embodiments, the colonic delivery system further comprises a metal compound, wherein the metal compound is incorporated into at least one of the plurality of polymer.

In other aspects, the colonic delivery system comprises a suppository base coated with a hydrophobic polymer, wherein the composition is a capsule and the substrate for chain elongation is dispersed in the suppository base. In some aspects, the colonic delivery system comprises a food component, wherein the colonic delivery system is specifically decomposed by an enzyme specific to the colon compared to other sites in the gastrointestinal tract. In certain embodiments, the food component is selected from the group consisting of: a non-starch polysaccharide, amylose, xanthan gum, and an azopolymer.

In still other aspects, the colonic delivery system comprises a liquid, semi-solid, or solid core and an outer shell layer. The liquid, semi-solid, or solid core contains the substrate for chain elongation, and the outer shell layer encapsulates the liquid, semi-solid, or solid core. In some embodiments, the outer shell layer ensures colonic delivery of the substrate for chain elongation. In other embodiments, the colonic delivery system further comprises a modified release coating that is applied to the outer shell layer. In such embodiments, the modified release coating ensures colonic delivery of the substrate for chain elongation.

In some embodiments, the concentration of the substrate for chain elongation in the composition is at least 1 mM, for example, 1 mM-120 mM. Where the substrate for chain elongation is ethanol, the concentration of substrate for chain elongation in the composition is 4 mM-100 mM. In some implementations, the concentration of ethanol for chain elongation in the composition is 40 mM-100 mM, 4 mM-40 mM, 4 mM-20 mM, 10 mM-20 mM, or 10 mM-15 mM. Where the substrate for chain elongation is lactate, the concentration of substrate for chain elongation in the composition is 1 mM-120 mM. In some implementations, the concentration of lactate in the composition is 1 mM-100 mM, 1 mM-40 mM, 1 mM-100 mM, 4 mM-100 mM, or 4 mM-40 mM.

Also disclosed are methods of increasing production of medium-chain and/or small chain fatty acids in the digestive tract of a subject and methods of ameliorating the symptoms of diabetes, obesity, Alzheimer's disease, Parkinson's disease, Amyotrophic Lateral Sclerosis (ALS), inflammatory bowel diseases (ulcerative colitis, UC, and Crohn's disease, CD), cancer (particularly colon cancer), and/or cardiomyopathies in a subject. These methods comprise administering to the subject the disclosed composition comprising ethanol, lactate, or a combination thereof in a colonic delivery system.

The foregoing and other aspects, features, and advantages will be apparent from the DESCRIPTION and DRAWINGS, and from the CLAIMS if any are included.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts, in accordance with certain embodiments, known microbial pathways for production of butyrate in the human colon.

FIG. 2 depicts, in accordance with certain embodiments, a proposed pathway for production of butyrate (and caproate).

FIGS. 3A-3D depict, in accordance with certain embodiments, a timeline of the conversion of acetate, lactate, and/or ethanol into metabolites via chain elongation across a period of 7 days.

FIG. 4 depicts, in accordance with certain embodiments, a timeline of methane production after administration of acetate, lactate, and/or ethanol over the course of 7 days.

FIGS. 5A-5G depict, in accordance with certain embodiments, a timeline of the conversion of acetate, lactate, and/or ethanol into metabolites via chain elongation across a period of 7 days.

FIGS. 6A-6P depict, in accordance with certain embodiments, a timeline of the conversion of acetate, lactate, and/or ethanol into metabolites via chain elongation across a period of 7 days. The results compare under conditions mimicking the pH environment of the ascending colon (pH 5.5) and the descending colon (pH 6.5).

### DESCRIPTION OF THE INVENTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The disclosure relates to the discovery that anaerobic microbial metabolism of chain elongation is a mechanism for butyrate and caproate production in the human colon. The examples confirm that microbes from the human colon can produce butyrate and caproate through chain elongation, an understudied pathway for gut microbes. The disclosure also relates to the identification of ideal conditions of chain elongation that leads to enhanced production of butyrate and caproate. Acetate, lactate, and ethanol, naturally found in the gut, are key substrates for acetate's elongation to butyrate and caproate. These discoveries opens new avenues for treatments metabolic and neurological disorders via microbiome modulation.

For example, new avenues of treatments metabolic and neurological disorders (including, but not limited to diabetes, obesity, Alzheimer's disease, Parkinson's disease, Amyotrophic Lateral Sclerosis (ALS), inflammatory bowel diseases (ulcerative colitis, UC, and Crohn's disease, CD), cancer (particularly colon cancer), and/or cardiomyopathies) may be increasing production of medium-chain and/or small chain fatty acids in the digestive tract of a subject. In one aspects, the method comprises administering to the subject a composition comprising a substrate for chain elongation by gut microbiome, for example lactate and/or ethanol. In some aspects, the composition comprises acetate and/or butyrate in addition to lactate and/or ethanol. The composition may be administered orally or rectally. In particular embodiments, the composition is administered orally in via a colonic delivery system.

A variety of pharmaceutical preparations that enable the delivery of a therapeutic agent to the colon (referred to herein as “a colonic delivery system”) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.5 and about 7.5. This range fall within the pH range in which the pH shifts toward the alkaline side at a distal portion of the stomach, which makes it a suitable range for use in the delivery to the colon. Additional pH-based colonic delivery systems are described in U.S. Pat. Nos. 5,482,718, 5,541,171, 5,716,648, and 5,866,619, the contents of which are incorporated by reference herein.

Another embodiment of a colonic delivery system is based on delaying the release of the contents by approximately 3 to 6 hours after gastric emptying, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Examples of the composition enabling the delivery to the colon further include bioadhesive compositions which specifically adhere to the colonic mucosal membrane (for example, a polymer described in the specification of U.S. Pat. No. 6,368,586, the contents of which are incorporated herein by this reference) and compositions into which a protease inhibitor is incorporated for protecting particularly a biopharmaceutical preparation in the gastrointestinal tracts from decomposition due to an activity of a protease. An exemplary bioadhesive composition comprises a polymer having a metal compound incorporated therein, which increases adherence to mucosal surfaces. In some aspects, the metal compound incorporated into the polymer is a water-insoluble metal compound, such as a water-insoluble metal oxide or hydroxide.

A further example of a colonic delivery system is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

Another example of a colonic delivery system is a system designed to be specifically decomposed by an enzyme specific to the colon compared to other sites in the gastrointestinal track (for example, a carbohydrate hydrolase or a carbohydrate reductase). Such a system is not particularly limited, and more specific examples thereof include systems which use food components such as non-starch polysaccharides, amylose, xanthan gum, and azopolymers.

Combinations of these known colonic delivery systems may be used in the oral compositions described herein. Colonic delivery systems utilizing a combination of the above described colonic delivery strategies are described, for example, in U.S. Pat. Nos. 6,773,720, 9,114,071, and 9,463,163, the contents of which are incorporated by reference herein. In a particular embodiment, the colonic delivery system in the disclosed composition comprises a plurality of polymers, wherein at least one polymer of the plurality of polymers prevent penetration of gastric fluid into the colonic delivery system and the plurality of polymers further comprises a pH-independent erodible polymer that delays the release of the at least one substrate for chain elongation for at least four hours after gastric emptying. In another embodiment, the colonic delivery system comprises a liquid, semi-solid, or solid core and an outer shell layer. The liquid, semi-solid, or solid core contains the substrate for chain elongation, and the outer shell layer encapsulates the liquid, semi-solid, or solid core. In some embodiments, the outer shell layer ensures colonic delivery of the substrate for chain elongation. In other embodiments, the colonic delivery system further comprises a modified release coating that is applied to the outer shell layer. In such embodiments, the modified release coating ensures colonic delivery of the substrate for chain elongation.

Accordingly, in some aspects, the oral compositions described herein is formulated for colonic delivery and comprises a substrate for chain elongation by a gut microbiome, wherein the substrate for chain elongation is selected from ethanol, lactate, or a combination thereof. The concentration of the substrate for chain elongation in the composition is in an amount sufficient to increase chain elongation by anaerobic microbes in the human colon without causing physiological harm. In some aspects, for example with respect to lactate, the concentration of the substrate for chain elongation does not exceed the levels of the substrates found in fecal samples. In infants, lactate was detected from low mM (close to 0 mM to more than 100 mM). In adults, the typical detected concentration is on around 4 mM or less but can also be as high as around 40 mM. for some substrates that are rapidly absorbed in the large intestine, such as ethanol, the concentration of the substrate without causing physiological harm may be higher than those found in the fecal samples. For example, the concentration of ethanol in the portal vein can be higher than 100 mM, but the concentration in fecal sample could be low or undetectable. Ethanol production by gut microbes has been reported to be in the range of 10 mM-15 mM, so the composition may comprise ethanol concentration as high as over 115-120 mM.

In some embodiments, the concentration of the substrate for chain elongation in the composition is at least 1 mM, for example, 1 mM-120 mM. Where the substrate for chain elongation is ethanol, the concentration of substrate for chain elongation in the composition is 4 mM-100 mM. In some implementations, the concentration of ethanol for chain elongation in the composition is 40 mM-100 mM, 4 mM-40 mM, 4 mM-20 mM, 10 mM-20 mM, or 10 mM-15 mM. Where the substrate for chain elongation is lactate, the concentration of substrate for chain elongation in the composition is 1 mM-120 mM. In some implementations, the concentration of lactate in the composition is 1 mM-100 mM, 1 mM-40 mM, 4 mM-100 mM, or 4 mM-40 mM.

As the Examples show that production of short-chain fatty acids (SCFAs) and medium-chain fatty acids (MCFAs) by the microbiome in the colon from administration of ethanol or lactate, methods of increasing production of SCFAs and/or MCFAs in the digestive tract of a subject is disclosed. The method comprises administering to the subject the composition comprising lactate and/or ethanol, such as those described above. As the administration of such compositions increases the production of SCFAs and MCFAs in the colon, methods of ameliorating the symptoms of diabetes, obesity, Alzheimer's disease, Parkinson's disease, ALS, inflammatory bowel diseases (UC or CD), cancer, and/or cardiomyopathies in a subject are also disclosed. In one aspects, the method comprises administering to the subject a composition comprising lactate, and/or ethanol, such as those described above. In some implementations, the compositions described herein with a colonic delivery system are orally administered to the subject.

### 1. Overview

Medium-chain fatty acids (MCFAs) serve not only as an energy source but also regulate glucose and lipid metabolism (Huang et al., 2021). Owing to their unique and rapid metabolic properties, MCFAs have been recognized as potential therapeutics for the treatment of diseases such as diabetes, obesity, epilepsy, Alzheimer's disease, and other metabolic disorders (Papamandjaris et al., 1998; Takikawa et al., 2013). Although they serve as an energy source, MCFAs also function as signaling molecules to regulate various cellular processes. Passive MCFA uptake into cells allows energy production to meet cellular demands, and this is especially important in disorders that affect the cellular transport of metabolites, such as type II citrullinemia, CD36 deficiency, and GLUT-1 deficiency syndrome, as well as disorders that affect Long-Chain Fatty Acid (LCFA) metabolism, such as long-chain fatty acid oxidation disorder (LC-FAOD), very long chain acyl-CoA dehydrogenase deficiency (VLCAD), and familial chylomicronemia syndrome (Sklirou et al., 2021; Williams et al., 2018). In metabolic disorders such as obesity, diabetes, and hypertriglyceridemia, Medium Chain Fatty Acids (MCFAs), Medium Chain Triglycerides (MCTs) are mainly used to promote lipid metabolism and weight loss, and thus restore or improve metabolic balance (Huang et al., 2021).

Medium-chain triglycerides/MCFA have a favorable safety profile and are used to treat a variety of disorders such as benefits in skin care (Assunção et al., 2009), weight loss (Tsuji et al., 2001), cholesterol level maintenance (Shilling et al., 2013), immunomodulatory effects (Nurul-Iman et al., 2013), cardiovascular uses (Zakaria et al., 2011) and more recently in Alzheimer's disease (Augustin et al., 2018). Capric, caprylic and caproic acids reduced colon cancer cell viability by 70% to 90% and also produced anticancer effects by down-regulating cell cycle regulatory genes and up-regulating genes involved in apoptosis. Similar effects were observed in the human skin epidermoid carcinoma cell line (A-431) (Narayanan et al., 2015). Medium Chain Triglycerides (MCT), as well as their constituent MCFA was isolated from milk and skin lipids and have a direct antibacterial effect at high concentrations on a variety of pathogenic gram-positive and gram-negative microbes including Campylobacter jejuni, Listeria monocytogenes, and Clostridium perfringens and has been explored as an alternative to antibiotic use in animals (Fischer et al., 2012; Papavassilis et al., 1999). MCFAs improve insulin resistance, reverse steatosis and cardiac dysfunction in animal models, and improve glucose disposal in humans. MCFA offers a therapeutic advantage in the preservation of β-cell function as part of a preventative strategy against diabetes in at risk populations (Roopashree et al., 2021).

Evidence from human studies suggests that MCFAs have potentials to treat a wide range of neurological and metabolic disorders such as drug-resistant epilepsy, obesity, type 2 diabetes, nonalcoholic fatty liver disease (NAFLD), cancer, and Alzheimer's disease (Fan et al., 2022).

Besides, some case-control studies used GC-MS revealed the reduced level of MCFAs (pentanoate, hexanoate, heptanoate and octanoate etc.) in IBD patients (De Preter et al., 2015; Garner et al., 2007). MCFAs have previously been shown to suppress inflammation and ameliorate experimental colitis in mice by activating the peroxisome proliferator activated receptor (PPAR)-c. Both capric acid and lauric acid have shown the anti-inflammatory and anti-bacterial properties (M. Li et al., 2022).

As directly absorbable fatty acids, MCFAs facilitate quick energy supply, promote intestinal mucosal repair, and regulate inflammation (Jia et al., 2019a). However, several studies have demonstrated the pro-inflammatory activity of MCFAs. MCFAs regulate inflammation in human fetal intestinal epithelial cells by promoting IL-8 secretion induced by IL-1b and TNF-a (Andoh et al., 2000). The colon plays a pivotal role in the digestion and absorption of MCFAs. Moreover, MCFAs exhibit inherent antibacterial effects, and maintain gut microbiota balance by reducing pathogenic bacteria and promoting beneficial bacteria (Jia et al., 2019a). The role of MCFAs in patients suffering from UC and CD has been elucidated by various epidemiological investigations. Observations from a case-control study have demonstrated significantly reduced levels of lauric acid (C12:0) in the serum of patients with autoimmune diseases (Tsoukalas et al., 2019). Reduced levels of MCFAs are common among IBD patients, and proportions of MCFAs differ between CD and UC. MCFAs alleviates IBD symptoms by regulating inflammation and intestinal epithelial barrier (Wang, Huang, et al., 2018; Yan et al., 2023).

The medium chain fatty acids (MCFAs) (pentanoate, hexanoate, heptanoate, octanoate and nonanoate) were significantly decreased in patients with Crohn's disease (CD), Ulcerative colitis (UC) (De Preter et al., 2015).

The therapeutic potential of MCFAs has been demonstrated for several neurodegenerative diseases, such as Alzheimer's disease and Parkinson's disease; however, there is currently limited clinical evidence supporting MCFA-based treatments for ALS (Dunn, Steinert, et al., 2023).

It was found that MCFAs and their esterified forms can decrease pathogens while increasing probiotics. In addition, being recognized via specific receptors, MCFAs are capable of alleviating inflammation to a certain extent by regulating inflammation and immune-related pathways. MCFAs may also have a certain value to relieve intestinal allergy and inflammatory bowel disease (IBD). Unknown mechanism of various MCFA characteristics still causes dilemmas in the application, thus MCFAs are used generally in limited dosages and combined with short-chain organic acids (SOAs) to attain ideal results (Jia et al., 2019b). Up to now, we have found that MCFAs with various carbon chain lengths have different bactericidal effects against Gram-positive and Gram-negative bacteria. Moreover, naturally existing MCFAs including C6:0, C8:0, C10:0 and C12:0 have been discovered to have stronger antimicrobial activity than their monoglycerides and fatty alcohols (Thormar & Hilmarsson, 2007; Zentek et al., 2012). MCFAs regulate the composition of microbiota to a balance of probiotics and pathogenic microorganisms. Blends of MCFAs along with organic acids (OAs) increase the population of Lactobacillus spp. and Bifidobacterium spp. (Wang, Lu, et al., 2018), while decreasing the number of fecal E. coli and total aerobics (Long et al., 2018). MCFAs have essential impacts not only on the direct bactericidal response but also on the secretion of antimicrobial peptides. MCFAs with chain lengths of C6-C10 can upregulate gene expression of pBD2, pBD3, pEP2C, B-defensin 9,cathelicidin B1, and cathelicidin LL-37 in vitro (Jiang et al., 2013; Sunkara et al., 2012; X. Zeng et al., 2013).

The gut microbiota-derived SCFAs have shown a variety of biological effects on the host, such as anti-inflammatory, immunoregulatory, anti-obesity, anti-diabetes, anticancer, cardiovascular protective, hepatoprotective, and neuroprotective effects (Fernández et al., 2016; X. Li et al., n.d.). SCFAs play an important part in obesity and energy metabolism by regulating the appetite. For example, a study showed that the mechanism of SCFAs suppressing food intake was related to vagal afferent, and the efficacy was butyrate>propionate>acetate. Moreover, butyrate exerted an anorexigenic effect through activating vagal afferent neurons and their projection sites, such as nucleus tractus solitaries (NTS) neurons, and directly increasing Ca2+ concentration in nodose ganglion neurons (NGNs) (Goswami et al., 2018). SCFAs could protect cardiovascular system by decreasing atherosclerosis. For example, a study showed that butyrate inhibited the progression of diet-induced atherosclerosis by decreasing intestinal cholesterol absorption via regulating related transporters expression, such as Niemann-Pick C1-like 1 (Npc111, a major intestinal cholesterol transporter) and ATP-binding cassette (ABC) transporters G5 and G8 (Chen et al., 2018). SCFAs have been shown to inhibit bacterial growth and viability. A study showed that butyrate could enhance the antimicrobial activity of macrophages without tissue damaging inflammation (Schulthess et al., 2019). SCFAs show various bioactivities, such as anti-inflammatory and immunoregulatory effects, as well as preventive and therapeutic effects on several diseases, including obesity, cardiovascular disease, liver disease, diabetes mellitus, IBDs, diarrhea, constipation, neurodegenerative diseases, neuropsychiatric diseases, cancers, arthritis and periodontal disease, and so on (Xiong et al., 2022).

MCFAs have been linked to the treatment of both aging and neurodegenerative disease via their effects on metabolism. Through action on multiple disease-related pathways, MCFAs are emerging as compounds with notable potential to promote healthy aging and ameliorate neurodegeneration. MCFAs have been shown to stimulate autophagy and restore mitochondrial function, which are found to be disrupted in aging and neurodegeneration (Dunn, Zhang, et al., 2023). Compared to LCFAs, MCFAs are more rapidly metabolized and generate ketones more efficiently, allowing for a more palatable diet due to the higher carbohydrate content (Huttenlocher et al., n.d.). Medium chain fatty acids and the MCT diet improve cognitive performance of patients suffering from AD and mild cognitive impairment (Krikorian et al., 2012; Taylor et al., 2018a). In addition, MCFAs have been shown to improve mitochondrial function and promote autophagy, both of which are known to be dysregulated in aging and NDs (Rangaraju et al., 2019; Stavoe & Holzbaur, 2019). Importantly, MCFAs can cross the blood brain barrier (BBB) (Wlaź et al., 2012,2015), raising the possibility that MCFAs may also be used to treat conditions affecting the central nervous system, including AD, ALS, PD, and even some types of brain tumors (Castellano et al., 2014; Croteau et al., 2018; Manzo et al., 2018; Q. Zeng et al., n.d.). MCFAs may promote healthy aging by upregulating mitochondrial biogenesis and reducing oxidative stress. MCFAs may improve symptoms of Alzheimer's disease by promoting mitochondrial biogenesis. MCFAs may alleviate symptoms of Parkinson's disease by elevating ketone levels. MCFAs may promote healthy aging via upregulation of autophagy (Dunn, Zhang, et al., 2023).

Supplementation with MCFAs may be a promising therapy for increasing energy available for brain function and for improving cognition in individuals with AD and in those at higher risk of developing AD (ie, individuals with mild cognitive impairment) (Castro et al., 2023). Supplementation with MCFAs increases circulating levels of ketone bodies. MCFA intervention improves cognitive functioning, potentially by increasing the amount and availability of energy for the brain. This holds promise for MCFA supplementation, which may be complementary to other interventions that might slow the neurodegenerative process underlying cognitive impairment and prevent further decline, such as physical activity and brain training (Henderson et al., 2009; Rebello et al., 2015). The evidence reviewed also showed that supplements containing a mixture of MCFAs with different chain lengths (triglycerides containing caproic acid [C6], caprylic acid [C8], capric acid [C10], and lauric acid [C12]) produced a more powerful effect (improvement in memory and cognition, and larger increase in fasting ketone bodies) (de la Rubia Ortí et al., 2018; Efficacy of Adjunctive Extra Virgin Coconut Oil Use in Moderate to Severe Alzheimer's Disease, n.d.; Fortier et al., 2019; Ota et al., 2019; Rebello et al., 2015; Taylor et al., 2018b; Xu et al., 2020) than supplements containing solely caprylic triglycerides (Henderson et al., 2020; Ohnuma et al., 2016).

Currently, increasing attention is given to SCFAs with respect to their putative role in the pathogenesis of allergies, as well as autoimmune, metabolic, and neurological diseases (Tan et al., 2014). In the last two decades, the role of MCFAs as agonists of peroxisome proliferator-activated receptors has also been characterized (Liberato et al., 2012). Moreover, accumulating evidence indicates that SCFAs generated by the gut microbiota exert influence on food intake, thereby regulating energy homeostasis and body weight (Bä ckhed et al., 2004; Byrne et al., 2015; Ramakrishna, 2013). While increasing evidence indicates that the diseased heart suffers from energy deficiency, fueling the myocardial energy metabolism with MCFAs has been proposed as metabolic therapy for treating patients suffering from certain cardiomyopathies (Labarthe et al., 2008a; Schönfeld & Wojtczak, 2016).

SCFAs are microbial metabolites reported to be responsible for maintaining the immune homeostasis of the central nervous, urinary and respiratory systems (Ratajczak et al., 2019). Intestinal and extra-intestinal ailments, including inflammatory bowel disease, neuropathology, allergy, diabetes, & obesity, are developed when misbalance occurs between gut microbiota and gastrointestinal tract (Holmes et al., 2011; Putignani et al., 2016). SCFAs, specifically butyrate and propionate, have shown beneficial effects in curtailing. Various metabolic ailments like diabetes, obesity and inflammatory bowel disease (IBD) via modification of TFs (transcription factors) and activation of specific GPCRs (G protein-coupled receptors) (Puertollano et al., 2014). Utilization of SCFAs and anti-inflammatory medicines, prebiotics, probiotics, symbiotics or as pro-drugs in cancer, and as coated objects in improving the healing characteristics of mucosal post colonic surgery could open a new window of opportunity in the field of therapeutics (Rauf et al., 2022).

There has been only one study in a rat model of diabetes with MCFAs, in which it was shown that the addition of hexanoate as sole FA source to hearts perfused with glucose alone normalized contractile function (Chatham & Forder, n.d.). Collectively, studies that have examined the effects of MCFAs in animal models of hypertrophy, both using the ex vivo perfused heart model or in vivo, have reported some benefits on cardiac function or disease progression (Allard et al., 2007; Labarthe et al., 2005, 2008b).

### 2. Experimental Approach and Materials and Methods

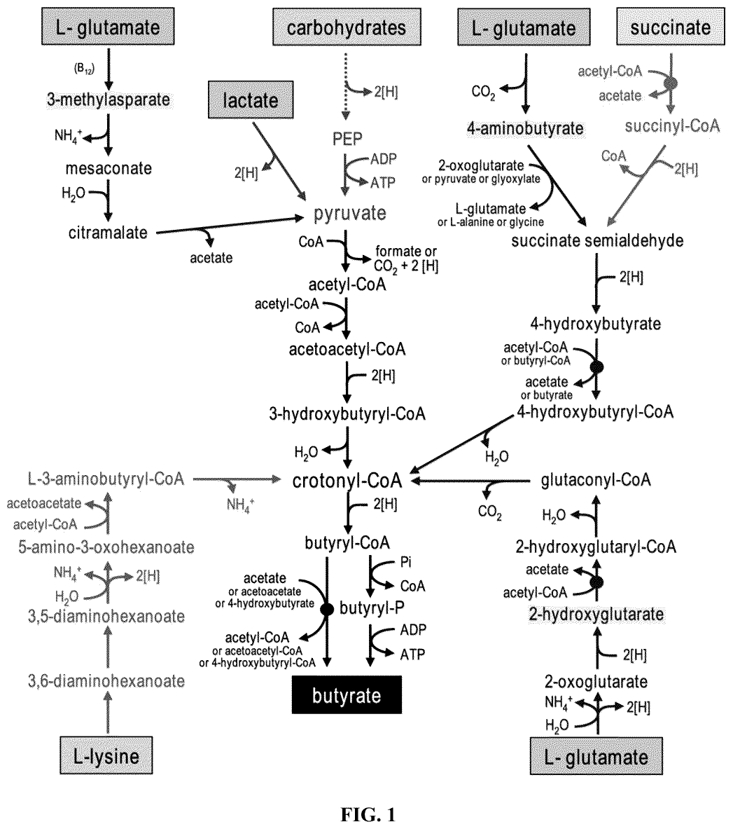

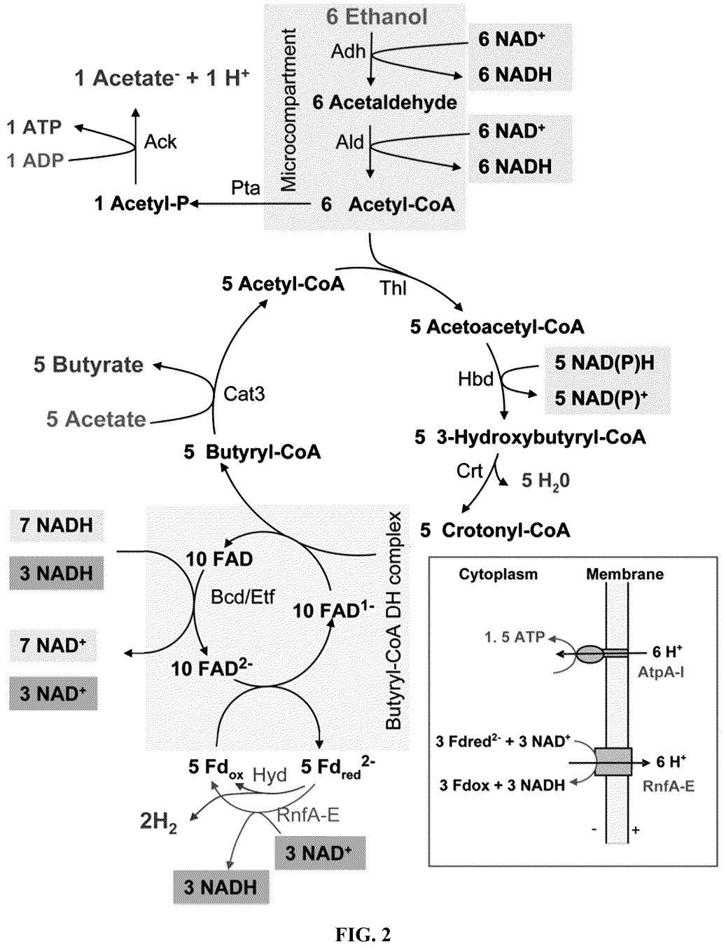

In microbial chain elongation, acetate, an abundant short-chain fatty acid in the colon, is elongated via reverse β-oxidation using a more reduced compound, such as ethanol or lactate to butyrate, caproate, and caprylate (C8). FIG. 1 depicts known microbial pathways for production of butyrate in the human colon. FIG. 2 depicts a proposed pathway for production of butyrate (and caproate).

In vitro bioreactors were setup using feces composited from multiple human donors. Gas composition in the bioreactors' headspace was analyzed for H₂, CO₂, and CH₄ using gas chromatography (GC). The concentrations of volatile fatty acids (VFAs), lactate, ethanol, were measured using high performance liquid chromatography (HPLC). 16S rRNA gene from Bacteria and Archaea was sequenced using the PacBio.

a. Medium Composition and Bioreactor Conditions

The ability of the human colon microbiome to carry out chain elongation reactions was probed in bioreactor experiments. Bioreactors consisted of 120 mL glass serum bottles containing 50 mL of reduced anaerobic mineral medium prepared using the Hungate technique. The medium contained the following stock solutions per liter: 10 mL of salts, 1 mL of trace element A, 1 mL of trace element B, and 1 mL of vitamin mix. The composition of the medium stock solutions was previously published (Robles et al. 2021) and is provided in the Tables 2-5. Sodium sulfide nonahydrate (0.2 mM) and L-cysteine (0.4 mM) were used as reducing agents. The medium was buffered with 15 mM 4-(2-hydroxyethyl)-1-piperazineethanesulfonic acid (HEPES) and 15 mM sodium bicarbonate. The medium pH was adjusted to 5.5 or 6.5 using a 10 N NaOH solution. The medium was bottled using a gas composition of 20% CO₂ and 80% N₂.

The experimental conditions and controls are shown in Table 1 and were tested in triplicate. The tested concentrations of short-chain fatty acids (60 mM acetate, 20 mM propionate, 20 mM butyrate) are within the typical range detected in the human colon. For ethanol and lactate (electron donors in chain elongation), we tested 4 mM, a low but physiological relevant concentration and a high concentration of 40 mM. The high concentration of ethanol (40 mM) was selected based on existing chain elongation literature (Yin et al. 2017; Coma et al. 2016; Steinbusch, Hamelers, and Buisman 2008; Robles et al. 2021; Joshi et al. 2021) and was strategically employed to enhance the activity of putative chain-elongating microorganisms. Each bioreactor was inoculated with 2 mL of a feces slurry (yielding 1000 mg feces per bioreactor). The slurry was prepared by mixing one-part reduced anaerobic mineral medium and one-part feces from a composite sample containing fecal samples from multiple healthy donors as described in the Supporting Information. In one condition, the fecal slurry was filtered through a 200 μm filter before inoculation. In condition with 60 mM acetate+40 mM ethanol+MCE culture at pH 6.5, a chain elongation culture derived from soil (Joshi et al. 2021) was also inoculated.

TABLE 1
Detailed conditions and concentrations of added substrates.

| | | | | Total | | Chain | |
|---|---|---|---|---|---|---|---|
| | | | | added | Anaerobic | elongation | Fecal |
| | Acetate | Ethanol | Lactate | carbon | medium | culture | matter |
| Condition | (mM) | (mM) | (mM) | (mM) | (mL) | (mL) | (mg) |
| 60 mM acetate + 40 mM ethanol | 60 | 40 | 0 | 200 | 50 | 0 | 1000 |
| 60 mM acetate + 40 mM ethanol + filtered feces | 60 | 40 | 0 | 200 | 50 | 0 | 1000 |
| 60 mM acetate + 40 mM ethanol + MCE culture | 60 | 40 | 0 | 200 | 50 | 0.5 | 1000 |
| 60 mM acetate + 4 mM ethanol | 60 | 4 | 0 | 128 | 50 | 0 | 1000 |
| 60 mM acetate | 60 | 0 | 0 | 120 | 50 | 0 | 1000 |
| 60 mM acetate + 40 mM ethanol (abiotic control) | 60 | 40 | 0 | 200 | 50 | 0 | 0 |
| 60 mM acetate + 40 mM lactate | 60 | 0 | 40 | 240 | 50 | 0 | 1000 |
| 60 mM acetate + 4 mM lactate | 60 | 0 | 4 | 132 | 50 | 0 | 1000 |
| 60 mM acetate + 40 mM lactate (abiotic control) | 60 | 0 | 40 | 240 | 50 | 0 | 0 |
| Background incubation control | 0 | 0 | 0 | 0 | 50 | 0 | 1000 |

b. Gas and Liquid Chemical Analyses

The pH was measured using 0.5 mL samples using an Orion™ Versa Star Pro™ Benchtop pH Meter (Thermo Scientific, Waltham, MA, USA) equipped with an Orion economy series pH electrode. The pH meter was calibrated with Orion pH 4.01, 7.00, and 10.01 standard solutions. Samples for HPLC consisted of 0.5 mL of supernatant from bioreactors filtered through a 0.2 μm PDVF filter (MDI Membrane Technologies, Harrisburg, PA, USA). The concentrations of formate, acetate, propionate, isobutyrate, butyrate, isovalerate, valerate, isocaproate, caproate, heptanoate, lactate, ethanol, butanol, and hexanol were determined using a high-performance liquid chromatograph (HPLC, Agilent, Santa Clara, CA, USA) with a photodiode array detector (DAD) set at 210 nm and a refractive index detector (RID). The HPLC was fitted with an Aminex HPX-87H column 300×7.8 mm (Bio-Rad Laboratories, Hercules, CA, USA) and operated with an eluent of 2.5 mM H₂SO₄ at a flow rate of 0.6 mL per minute for a duration of 80 minutes. The oven temperature was constant at 15° C. The calibration range for fatty acids was 0.25-10 mM, and the calibration range for the alcohols was 2.5-50 mM.

Gas samples (200 μL) were taken from the headspace of the bioreactors to measure H₂, CO₂, and methane using a gas chromatograph (GC, Shimadzu GC 2010, Kyoto, Japan) with a thermal conductivity detector (TCD) fitted with a Carboxen 1010 PLOT column 30 m×0.53 mm (Supelco, Bellefonte, PA, USA). Ultra-high purity argon was used as the carrier gas to maintain a constant pressure and flow rate of 55.3 kPa and 38.1 mL min⁻¹, respectively. The temperature of the injector was 200° C., the TCD temperature was 230° C., and the current was set at 41 mA. The column temperature was initially held to 60° C. for 3.2 min, ramped to 210° C. at 75° C.min⁻¹, and held for 1.4 min. The total gas volume in the headspace of the bioreactors was measured using a 50-mL frictionless syringe (Perfektum R matched numbered hypodermic syringes, Sigma Aldrich, St. Louis, MO, USA).

TABLE 2
Composition of 1 L of salt stock solution
used for medium preparation.

| Compound | Amount (g) |
|---|---|
| NaCl | 10 |
| MgCl₂ × 6H₂O | 5 |
| KH₂PO₄ | 20 |
| NH₄Cl | 30 |
| KCl | 30 |
| CaCl₂ × 2H₂O | 0.5 |

TABLE 3
Composition of 1 L of trace element solution
A used for medium preparation.

| Compound | Amount | |
|---|---|---|
| HCl (25% solution) | 10 | mL |
| FeCl₂ × 4H₂O | 1.5 | g |
| CoCl₂ × 6H₂O | 0.19 | g |
| MnCl₂ × 4H₂O | 0.1 | g |
| ZnCl₂ | 70 | mg |
| H₃BO₃ | 6 | mg |
| Na₂MoO₃ × 2H₂O | 36 | mg |
| NiCl₂ × 6H₂O | 24 | mg |
| CuCl₂ × 2H₂O | 2 | mg |

TABLE 4
Composition of 1 L of trace element solution
B used for medium preparation.

| Compound | Amount (mg) |
|---|---|
| Na₂SeO₃ × 5H₂O | 6 |
| Na₂WO₄ × 2H₂O | 8 |
| NaOH | 500 |

TABLE 5
Composition of 1 L of vitamin solution.

| Compound | Amount (mg) |
|---|---|
| Folic acid | 20 |
| Pyridoxine hydrochloride | 100 |
| Riboflavin | 50 |
| Biotin | 20 |
| Thiamine | 50 |
| Nicotinic acid | 50 |
| Calcium Pantothenate | 50 |
| Vitamin B12 | 1 |
| p-Aminobenzoic acid | 50 |
| Thioctic acid | 50 |

c. Fecal Slurry Preparation

To prepare the fecal slurry for inoculation of bioreactors, a frozen composite fecal sample was thawed out in the refrigerator overnight. Then, in an anaerobic glove chamber, we combined one part of the thawed sample with one part anaerobic mineral medium (no carbon source added) in a serum bottle. The slurry was then incubated at a temperature of 37° C. overnight. At time 0 (beginning of experiment), each bioreactor was inoculated with 2 mL fecal slurry.

### 3. Results

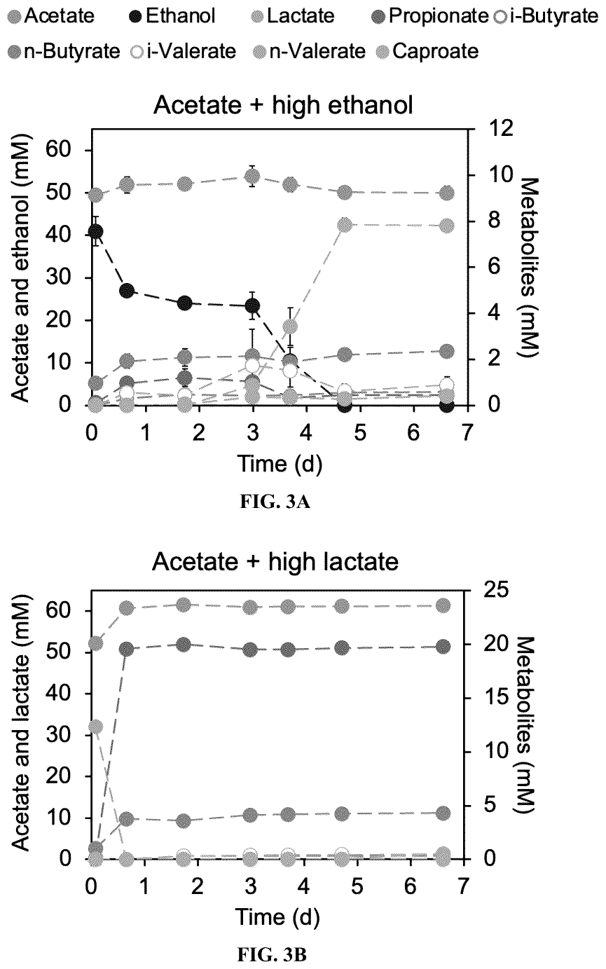

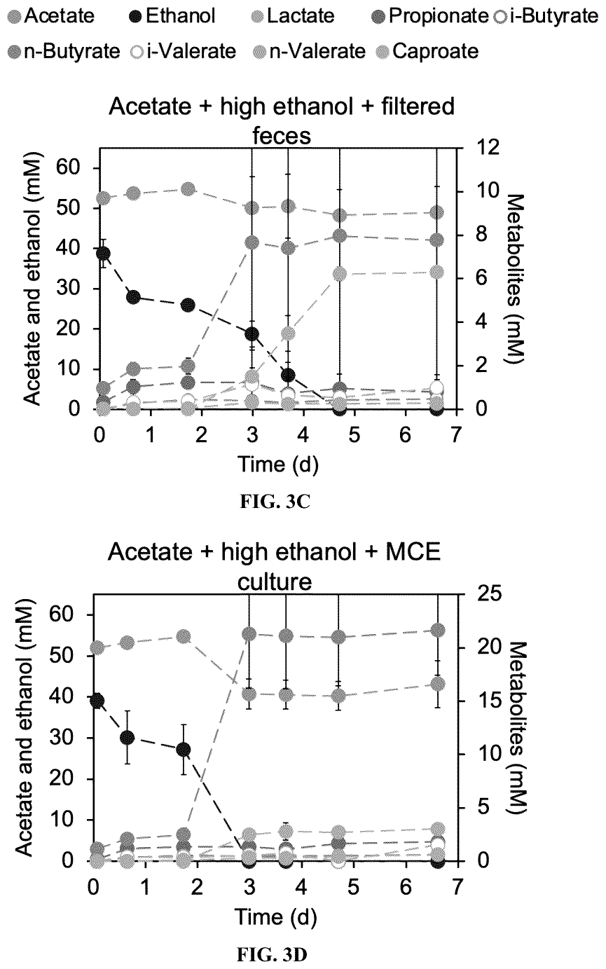

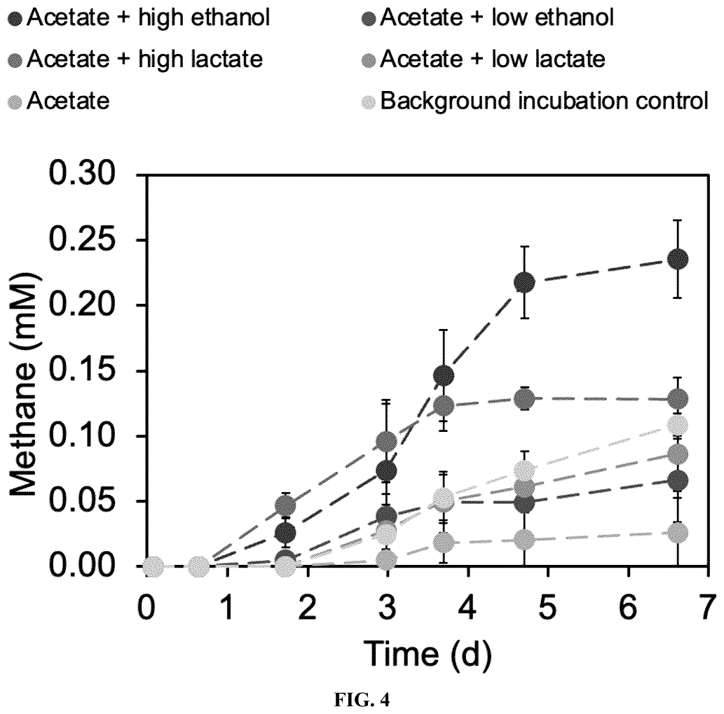

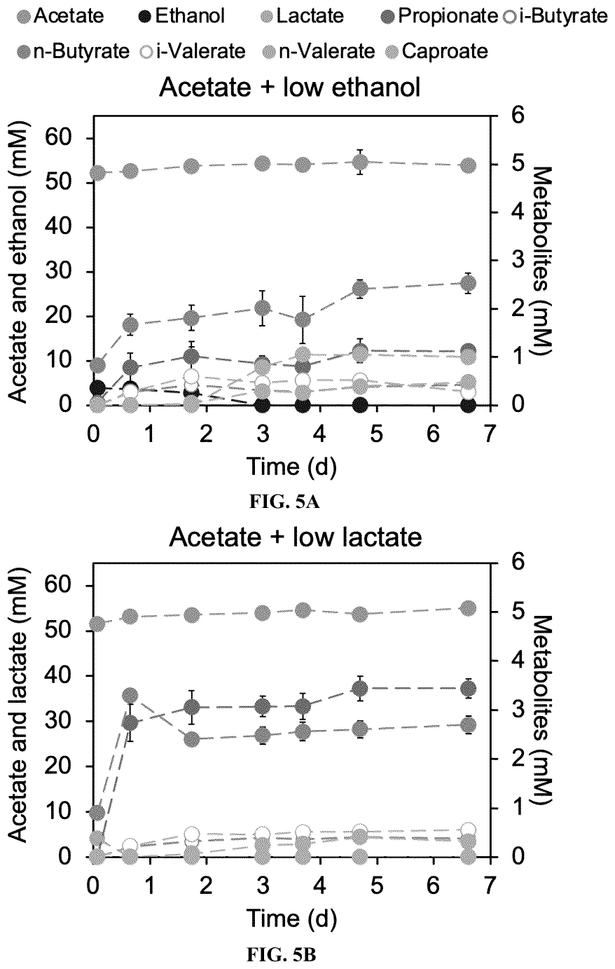

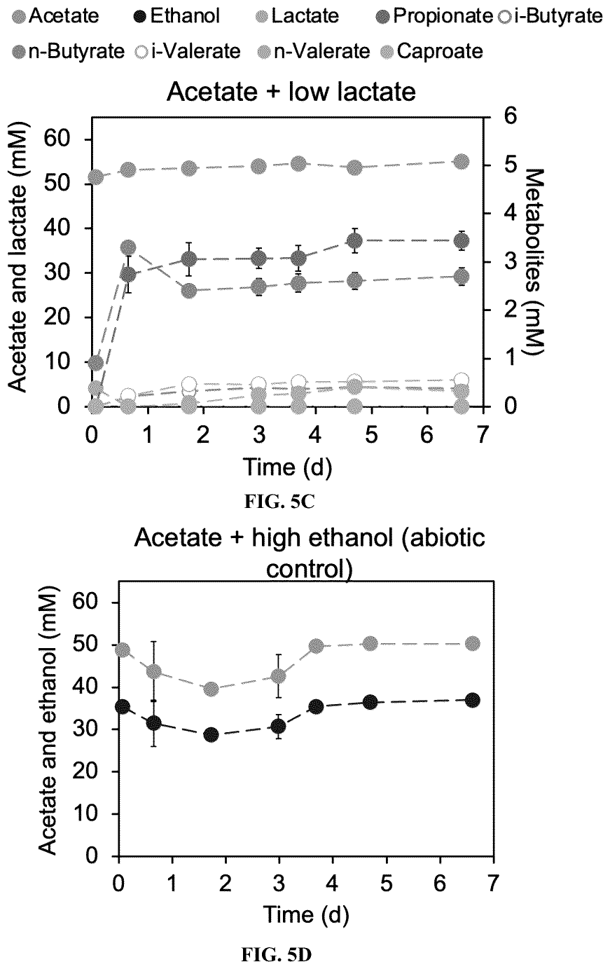

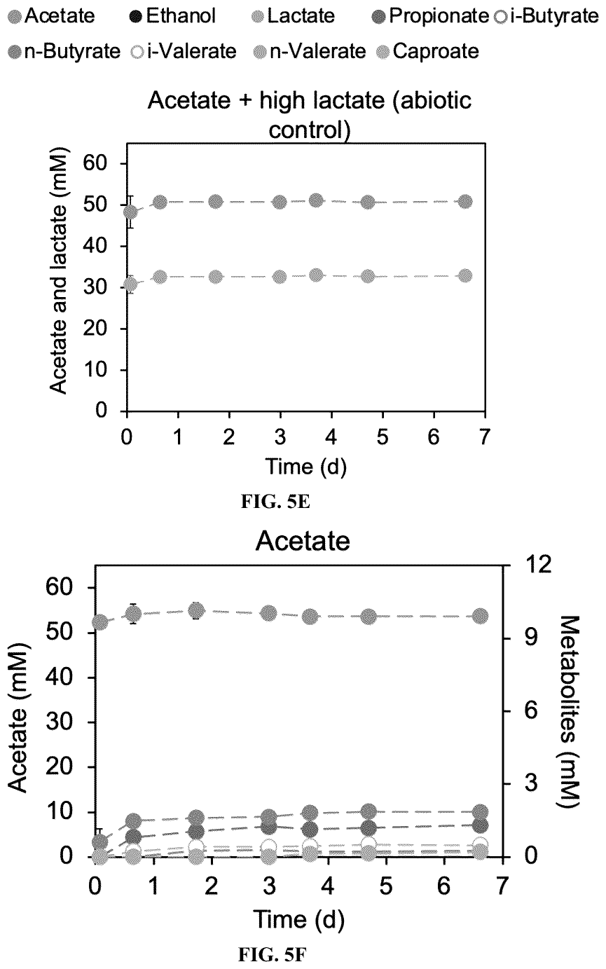

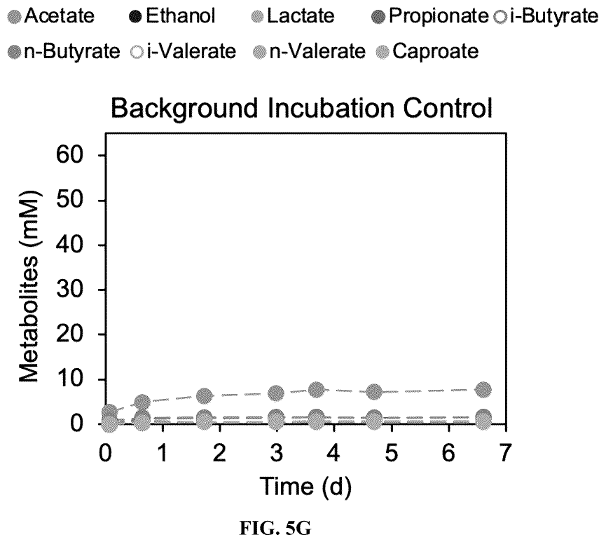

Conversion of acetate and ethanol to butyrate and caproate via chain elongation occurred within one day and resulted in production of these metabolites (FIGS. 3A and 3B). With lactate as the electron donor, a metabolic shift was observed towards propionate and butyrate production (FIG. 3B). Filtering the feces to remove larger particles did not affect the chain elongation activity, maintaining butyrate and caproate as the major metabolites (FIGS. 3C and 3D). Methane concentrations were higher at higher concentrations of ethanol and lactate but low overall relative to VFA metabolites (FIG. 4). Butyrate production via chain elongation also occurred at physiologically relevant concentration of ethanol and lactate (FIGS. 5A and 5B), although caproate was produced only when ethanol was the electron donor. As expected, in the absence of feces/microbial inoculum, consumption of added substrates was absent (FIG. 5C). Without added ethanol, lactate, or acetate, butyrate production was significantly lower. Acetate, propionate, and butyrate production in the absence of added substrates is likely through fermentation of fecal material components.

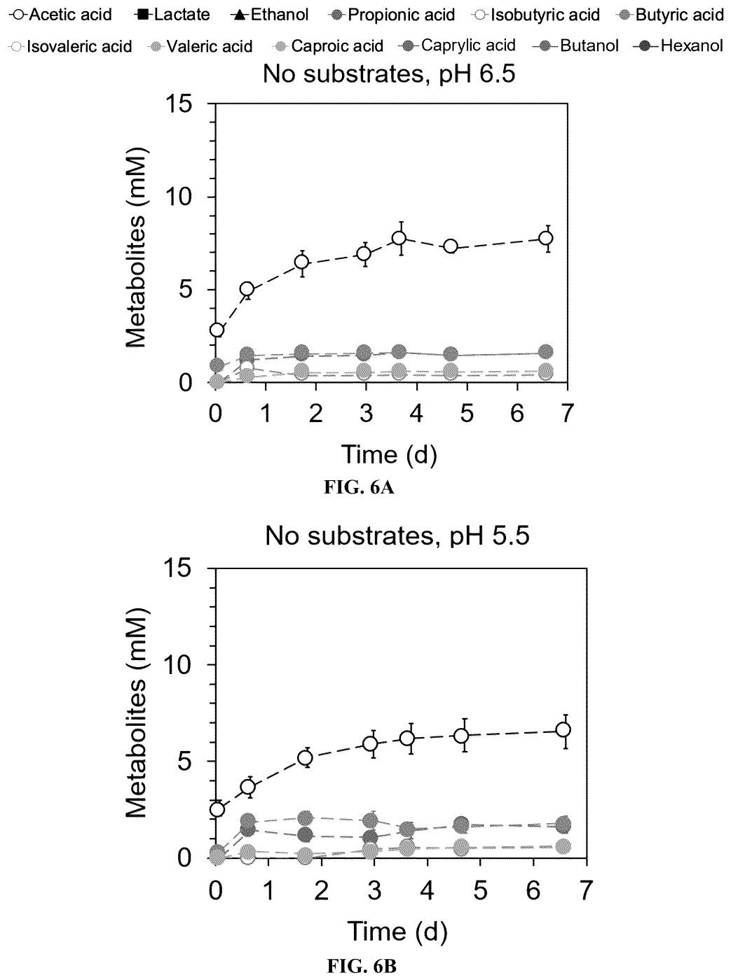

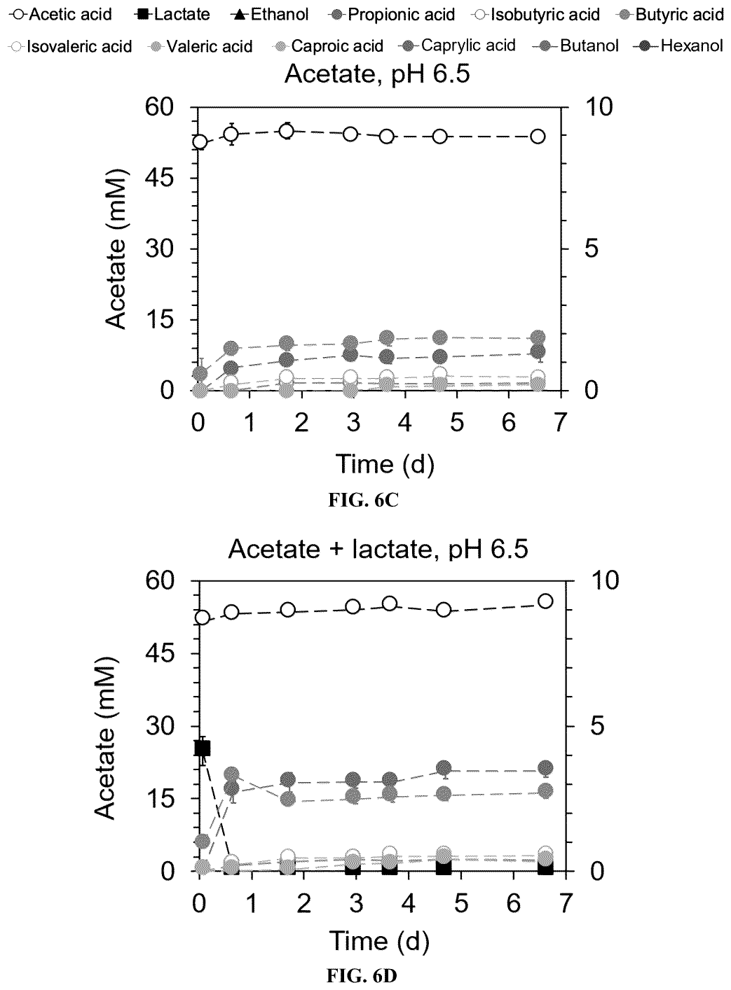

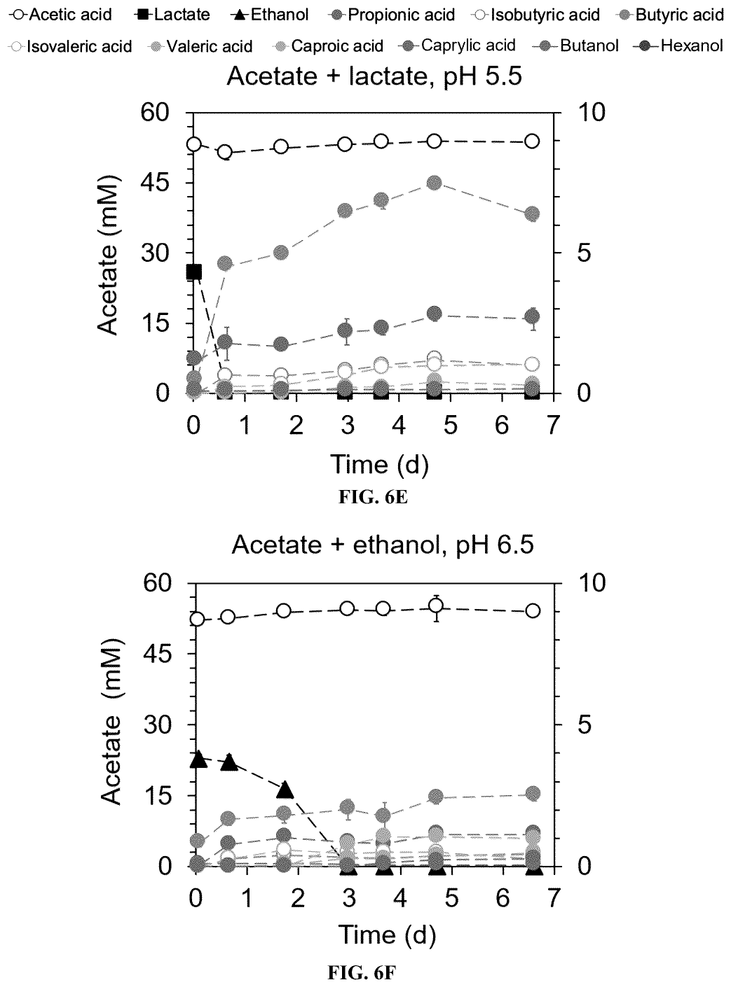

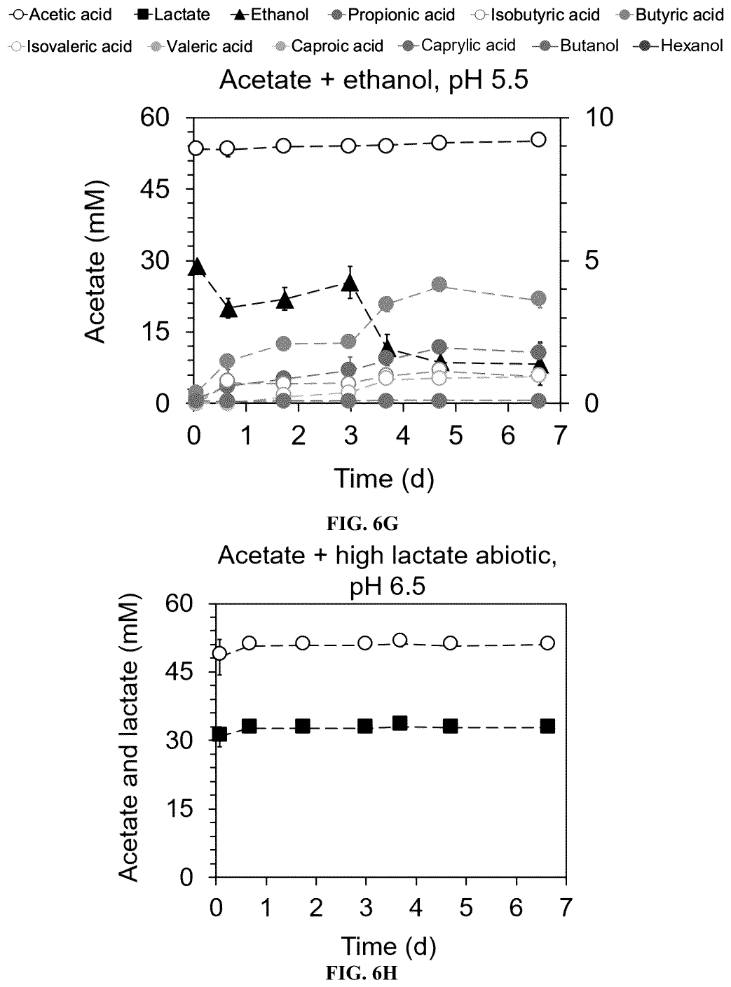

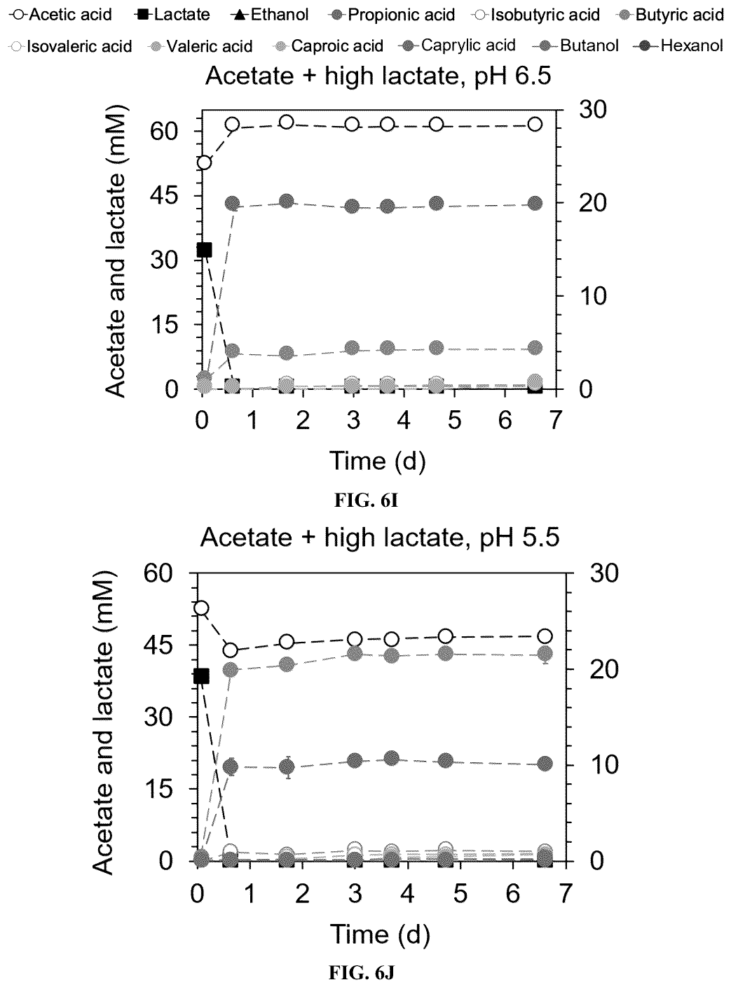

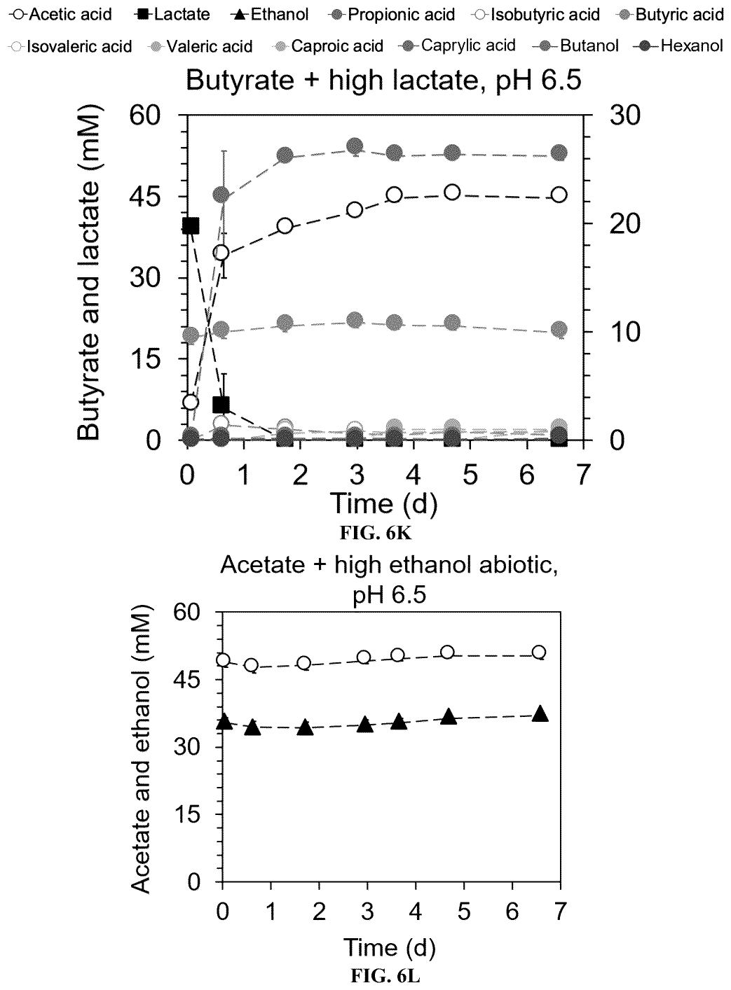

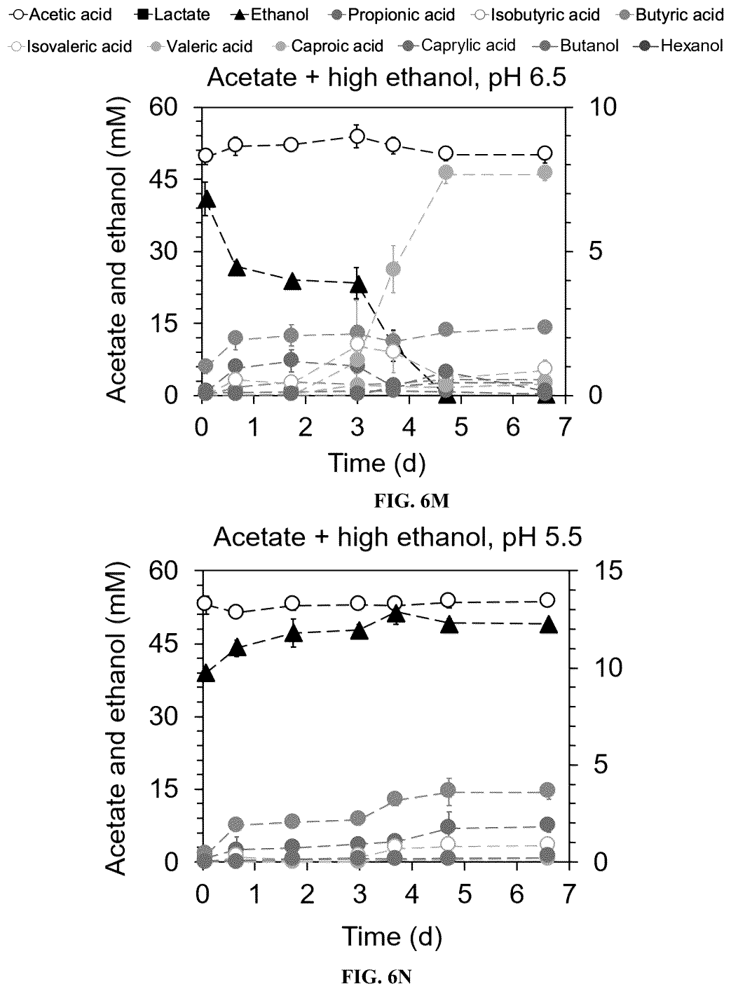

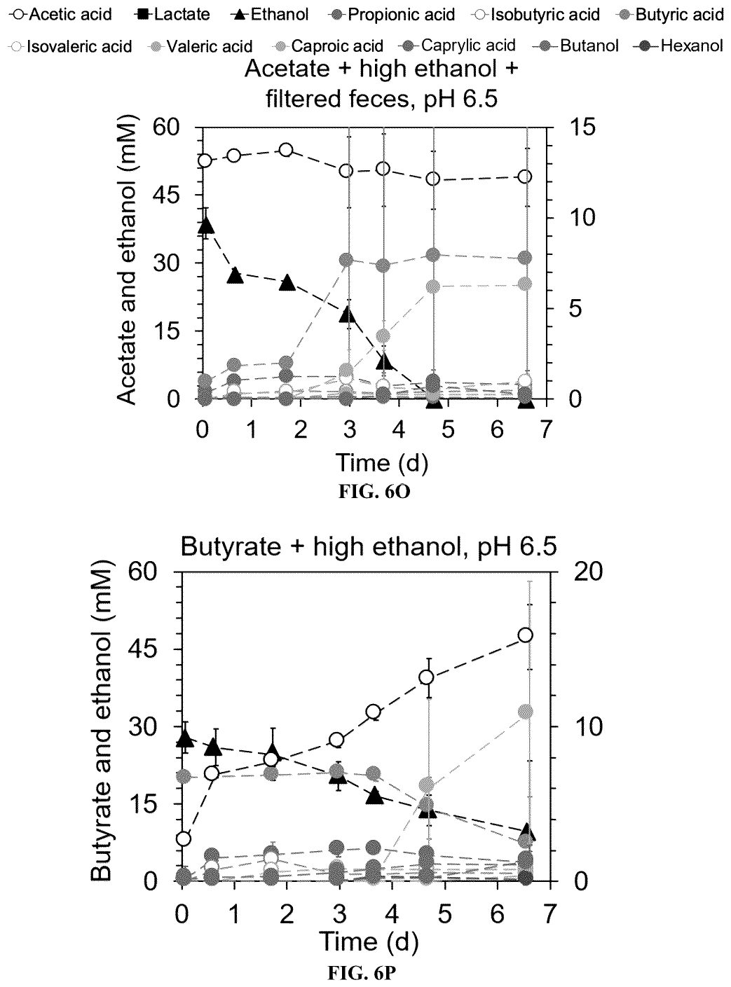

As shown in FIGS. 6A-6P, ethanol and lactate as substrates for chain elongation from microbes in the gut microbiome successfully increases SCFA and MCFA production in the varied conditions of the large intestine. The pH of the ascending colon is 5.5, and lactate on the whole is a better substrate for production of butyrate and other SCFAs via chain elongation than acetate or ethanol. The pH of the descending colon is 6.5, and higher concentrations of both lactate and ethanol showed significant increase in the production of MCFAs.

Many additional implementations are possible. Further implementations are within the CLAIMS.

### REFERENCES CITED AND INCORPORATED BY REFERENCE

Allard, M. F., Parsons, H. L., Saeedi, R., Wambolt, R. B., Brownsey, R., & Allard, M. F. (2007). AMPK and metabolic adaptation by the heart to pressure overload. Am J Physiol Heart Circ Physiol, 292, 140-148. https://doi.org/10.1152/ajpheart.00424.2006.-Ac
Andoh, A., Takaya, H., Araki, Y., Tsujikawa, T., Fujiyama, Y., & Bamba, T. (2000). Medium-and Long-Chain Fatty Acids Differentially Modulate Interleukin-8 Secretion in Human Fetal Intestinal Epithelial Cells. The Journal of Nutrition, 130 (11), 2636-2640. https://doi.org/10.1093/JN/130.11.2636.
Assunção, M. L., Ferreira, H. S., Dos Santos, A. F., Cabral, C. R., & Florêncio, T. M. M. T. (2009). Effects of dietary coconut oil on the biochemical and anthropometric profiles of women presenting abdominal obesity. Lipids, 44 (7), 593-601. https://doi.org/10.1007/s11745-009-3306-6
Augustin, K., Khabbush, A., Williams, S., Eaton, S., Orford, M., Cross, J. H., Heales, S. J. R., Walker, M. C., & Williams, R. S. B. (2018). Mechanisms of action for the medium-chain triglyceride ketogenic diet in neurological and metabolic disorders. In The Lancet Neurology (Vol. 17, Issue 1, pp. 84-93). Lancet Publishing Group. https://doi.org/10.1016/S1474-4422 (17) 30408-8.
Bäckhed, F., Ding, H., Wang, T., Hooper, L. V, Young Koh, G., Nagy, A., Semenkovich, C. F., & Gordon, J. I. (2004). The gut microbiota as an environmental factor that regulates fat storage. www.pnas.orgcgidoi10.1073pnas.0407076101
Byrne, C. S., Chambers, E. S., Morrison, D. J., & Frost, G. (2015). The role of short chain fatty acids in appetite regulation and energy homeostasis. International Journal of Obesity, 39 (9), 1331-1338. https://doi.org/10.1038/ijo.2015.84
Castellano, C. A., Nugent, S., Paquet, N., Tremblay, S., Bocti, C., Lacombe, G., Imbeault, H., Turcotte, É., Fulop, T., & Cunnane, S. C. (2014). Lower brain 18F-fluorodeoxyglucose uptake but normal 11C-acetoacetate metabolism in mild Alzheimer's disease dementia. Journal of Alzheimer's Disease, 43 (4), 1343-1353. https://doi.org/10.3233/JAD-141074
Castro, C. B., Dias, C. B., Hillebrandt, H., Sohrabi, H. R., Chatterjee, P., Shah, T. M., Fuller, S. J., Garg, M. L., & Martins, R. N. (2023). Medium-chain fatty acids for the prevention or treatment of Alzheimer's disease: a systematic review and meta-analysis. In Nutrition Reviews (Vol. 81, Issue 9, pp. 1144-1162). Oxford University Press. https://doi.org/10.1093/nutrit/nuac104
Chatham, J. C., & Forder, J. R. (n.d.). Relationship between cardiac function and substrate oxidation in hearts of diabetic rats. www.physiology.org/journal/ajpheart.
Chen, Y., Xu, C., Huang, R., Song, J., Li, D., & Xia, M. (2018). Butyrate from pectin fermentation inhibits intestinal cholesterol absorption and attenuates atherosclerosis in apolipoprotein E-deficient mice. The Journal of Nutritional Biochemistry, 56, 175-182. https://doi.org/10.1016/J.JNUTBIO.2018.02.011
Coma, Marta, Ramiro Vilchez-Vargas, Hugo Roume, Ruy Jauregui, Dietmar H. Pieper, and Korneel Rabaey. 2016. “Product diversity linked to substrate usage in chain elongation by mixed-culture fermentation.” Environmental Science & Technology 50 (12): 6467-6476. doi: 10.1021/acs.est.5b06021.
Croteau, E., Castellano, C. A., Fortier, M., Bocti, C., Fulop, T., Paquet, N., & Cunnane, S. C. (2018). A cross-sectional comparison of brain glucose and ketone metabolism in cognitively healthy older adults, mild cognitive impairment and early Alzheimer's disease. Experimental Gerontology, 107, 18-26. https://doi.org/10.1016/J.EXGER.2017.07.004
de la Rubia Ortí, J. E., García-Pardo, M. P., Drehmer, E., Cantus, D. S., Rochina, M. J., Aguilar Calpe, M. A., & Yang, I. H. (2018). Improvement of Main Cognitive Functions in Patients with Alzheimer's Disease after Treatment with Coconut Oil Enriched Mediterranean Diet: A Pilot Study. Journal of Alzheimer's Disease, 65 (2), 577-587. https://doi.org/10.3233/JAD-180184.
De Preter, V., Machiels, K., Joossens, M., Arijs, I., Matthys, C., Vermeire, S., Rutgeerts, P., & Verbeke, K. (2015). Faecal metabolite profiling identifies medium-chain fatty acids as discriminating compounds in IBD. Gut, 64 (3), 447-458. https://doi.org/10.1136/gutjnl-2013-306423.
Dunn, E., Steinert, J. R., Stone, A., Sahota, V., Williams, R. S. B., Snowden, S., & Augustin, H. (2023). Medium-Chain Fatty Acids Rescue Motor Function and Neuromuscular Junction Degeneration in a Drosophila Model of Amyotrophic Lateral Sclerosis. Cells, 12 (17). https://doi.org/10.3390/cells12172163
Dunn, E., Zhang, B., Sahota, V. K., & Augustin, H. (2023). Potential benefits of medium chain fatty acids in aging and neurodegenerative disease. In Frontiers in Aging Neuroscience (Vol. 15). Frontiers Media SA. https://doi.org/10.3389/fnagi.2023.1230467
Efficacy of Adjunctive Extra Virgin Coconut Oil Use in Moderate to Severe Alzheimer's Disease. (n.d.).
Fan, L., Zhu, X., Sun, S., Yu, C., Huang, X., Ness, R., Dugan, L. L., Shu, L., Seidner, D. L., Murff, H. J., Fodor, A. A., Azcarate-Peril, M. A., Shrubsole, M. J., & Dai, Q. (2022). Ca:Mg ratio, medium-chain fatty acids, and the gut microbiome. Clinical Nutrition, 41 (11), 2490-2499. https://doi.org/10.1016/j.clnu.2022.08.031.
Fernández, J., Redondo-Blanco, S., Gutiérrez-del-Río, I., Miguélez, E. M., Villar, C. J., & Lombó, F. (2016). Colon microbiota fermentation of dietary prebiotics towards short-chain fatty acids and their roles as anti-inflammatory and antitumour agents: A review. Journal of Functional Foods, 25, 511-522. https://doi.org/10.1016/J.JFF.2016.06.032
Fischer, C. L., Drake, D. R., Dawson, D. V., Blanchette, D. R., Brogden, K. A., & Wertz, P. W. (2012). Antibacterial activity of sphingoid bases and fatty acids against gram-positive and gram-negative bacteria. Antimicrobial Agents and Chemotherapy, 56 (3), 1157-1161. https://doi.org/10.1128/AAC.05151-11
Fortier, M., Castellano, C. A., Croteau, E., Langlois, F., Bocti, C., St-Pierre, V., Vandenberghe, C., Bernier, M., Roy, M., Descoteaux, M., Whittingstall, K., Lepage, M., Turcotte, É. E., Fulop, T., & Cunnane, S. C. (2019). A ketogenic drink improves brain energy and some measures of cognition in mild cognitive impairment. Alzheimer's & Dementia, 15 (5), 625-634. https://doi.org/10.1016/J.JALZ.2018.12.017.
Garner, C. E., Smith, S., Lacy Costello, B., White, P., Spencer, R., Probert, C. S. J., & Ratcliffem, N. M. (2007). Volatile organic compounds from feces and their potential for diagnosis of gastrointestinal disease. The FASEB Journal, 21 (8), 1675-1688. https://doi.org/10.1096/fj.06-6927com
Goswami, C., Iwasaki, Y., & Yada, T. (2018). Short-chain fatty acids suppress food intake by activating vagal afferent neurons. The Journal of Nutritional Biochemistry, 57, 130-135. https://doi.org/10.1016/J.JNUTBIO.2018.03.009
Henderson, S. T., Morimoto, B. H., Cummings, J. L., Farlow, M. R., & Walker, J. (2020). A Placebo-Controlled, Parallel-Group, Randomized Clinical Trial of AC-1204 in Mild-To-Moderate Alzheimer's Disease. Journal of Alzheimer's Disease, 75 (2), 547-557. https://doi.org/10.3233/jad-191302
Henderson, S. T., Vogel, J. L., Barr, L. J., Garvin, F., Jones, J. J., & Costantini, L. C. (2009). Study of the ketogenic agent AC-1202 in mild to moderate Alzheimer's disease: A randomized, double-blind, placebo-controlled, multicenter trial. Nutrition and Metabolism, 6. https://doi.org/10.1186/1743-7075-6-31
Holmes, E., Li, J. V., Athanasiou, T., Ashrafian, H., & Nicholson, J. K. (2011). Understanding the role of gut microbiome-host metabolic signal disruption in health and disease. In Trends in Microbiology (Vol. In 19, Issue 7, pp 349-359). https://doi.org/10.1016/j.tim.2011.05.006
Huang, L., Gao, L., & Chen, C. (2021). Role of Medium-Chain Fatty Acids in Healthy Metabolism: A Clinical Perspective. In Trends in Endocrinology and Metabolism (Vol. 32, Issue 6, pp. 351-366). Elsevier Inc. https://doi.org/10.1016/j.tem.2021.03.002.
Huttenlocher, P. R., Wilbourn, A. J., & Signore, J. M. (n.d.). Medium-chain triglycerides as a therapy for intractable childhood epilepsy.
Jia, M., Zhang, Y., Gao, Y., & Ma, X. (2019a). Effects of Medium Chain Fatty Acids on Intestinal Health of Monogastric Animals. Current Protein & Peptide Science, 21 (8), 777-784. https://doi.org/10.2174/1389203721666191231145901
Jiang, W., Sunkara, L. T., Zeng, X., Deng, Z., Myers, S. M., & Zhang, G. (2013). Differential regulation of human cathelicidin LL-37 by free fatty acids and their analogs. Peptides, 50, 129-138. https://doi.org/10.1016/J.PEPTIDES.2013.10.008.
Joshi, S., A. Robles, S. Aguiar, and A. G. Delgado. 2021. “The occurrence and ecology of microbial chain elongation of carboxylates in soils.” ISME Journal 15 (7): 1907-1918. doi: 10.1038/s41396-021-00893-2.
Krikorian, R., Shidler, M. D., Dangelo, K., Couch, S. C., Benoit, S. C., & Clegg, D. J. (2012). Dietary ketosis enhances memory in mild cognitive impairment. Neurobiology of Aging, 33 (2), 425.e19-425.e27. https://doi.org/10.1016/J.NEUROBIOLAGING.2010.10.006.
Labarthe, F., Gélinas, R., & Des Rosiers, C. (2008a). Medium-chain fatty acids as metabolic therapy in cardiac disease. Cardiovascular Drugs and Therapy, 22 (2), 97-106. https://doi.org/10.1007/s10557-008-6084-0
Labarthe, F., Khairallah, M., Bouchard, B., Stanley, W. C., Des Rosiers, C., & Des, C. (2005). Fatty acid oxidation and its impact on response of spontaneously hypertensive rat hearts to an adrenergic stress: benefits of a medium-chain fatty acid. Am J Physiol Heart Circ Physiol, 288, 1425-1436. https://doi.org/10.1152/ajpheart.00722.2004.
Li, M., Yang, L., Mu, C., Sun, Y., Gu, Y., Chen, D., Liu, T., & Cao, H. (2022). Gut microbial metabolome in inflammatory bowel disease: From association to therapeutic perspectives. In Computational and Structural Biotechnology Journal (Vol. 20, pp. 2402-2414). Elsevier B.V. https://doi.org/10.1016/j.csbj.2022.03.038
Li, X., Shimizu, Y., & Kimura, I. (n.d.). Gut microbial metabolite short-chain fatty acids and obesity.
Liberato, M. V., Nascimento, A. S., Ayers, S. D., Lin, J. Z., Cvoro, A., Silveira, R. L., Martínez, L., Souza, P. C. T., Saidemberg, D., Deng, T., Amato, A. A., Togashi, M., Hsueh, W. A., Phillips, K., Palma, M. S., Neves, F. A. R., Skaf, M. S., Webb, P., & Polikarpov, I. (2012). Medium chain fatty acids are selective peroxisome proliferator activated receptor (PPAR) γ activators and Pan-PPAR partial agonists. PLOS ONE, 7 (5). https://doi.org/10.1371/journal.pone.0036297.
Long, S. F., Xu, Y. T., Pan, L., Wang, Q. Q., Wang, C. L., Wu, J. Y., Wu, Y. Y., Han, Y. M., Yun, C. H., & Piao, X. S. (2018). Mixed organic acids as antibiotic substitutes improve performance, serum immunity, intestinal morphology and microbiota for weaned piglets. Animal Feed Science and Technology, 235, 23-32. https://doi.org/10.1016/J.ANIFEEDSCI.2017.08.018
Louis, P., Duncan, H. S., Sheridan, P. O., Walker, A. W., & Flint, H. J. (2022). Microbial lactate utilisation and the stability of the gut microbiome. Gut Microbiome, 3, e3, 1-16.
Manzo, E., O'Conner, A. G., Barrows, J. M., Shreiner, D. D., Birchak, G. J., & Zarnescu, D. C. (2018). Medium-chain fatty acids, beta-hydroxybutyric acid and genetic modulation of the carnitine shuttle are protective in a drosophila model of ALS based on TDP-43. Frontiers in Molecular Neuroscience, 11. https://doi.org/10.3389/fnmol.2018.00182.
Meijnikman, A. S., Davids, M., Herrema, H., et al. (2022). Microbiome-derived ethanol in nonalcoholic fatty liver disease. Nat Med 28, 2100-2106. https://doi.org/10.1038/s41591 -022-02016-6
Narayanan, A., Baskaran, S. A., Amalaradjou, M. A. R., & Venkitanarayanan, K. (2015). Anticarcinogenic properties of medium chain fatty acids on human colorectal, skin and breast cancer cells in vitro. International Journal of Molecular Sciences, 16 (3), 5014-5027. https://doi.org/10.3390/ijms16035014
Nurul-Iman, B. S., Kamisah, Y., Jaarin, K., & Qodriyah, H. M. S. (2013). Virgin coconut oil prevents blood pressure elevation and improves endothelial functions in rats fed with repeatedly heated palm oil. Evidence-Based Complementary and Alternative Medicine, 2013. https://doi.org/10.1155/2013/629329
Ohnuma, T., Toda, A., Kimoto, A., Takebayashi, Y., Higashiyama, R., Tagata, Y., Ito, M., Ota, T., Shibata, N., & Arai, H. (2016). Benefits of use, and tolerance of, medium-chain triglyceride medical food in the management of Japanese patients with Alzheimer's disease: A prospective, open-label pilot study. Clinical Interventions in Aging, 11, 29-36. https://doi.org/10.2147/CIA.S95362
Ota, M., Matsuo, J., Ishida, I., Takano, H., Yokoi, Y., Hori, H., Yoshida, S., Ashida, K., Nakamura, K., Takahashi, T., & Kunugi, H. (2019). Effects of a medium-chain triglyceride-based ketogenic formula on cognitive function in patients with mild-to-moderate Alzheimer's disease. Neuroscience Letters, 690, 232-236. https://doi.org/10.1016/J.NEULET.2018.10.048.
Papamandjaris, A. A., Macdougall, D. E., & Jones, P. J. H. (1998). Medium chain fatty acid metabolism and energy expenditure: Obesity treatment implications. Life Sciences, 62 (14), 1203-1215. https://doi.org/10.1016/S0024-3205 (97) 01143-0
Papavassilis, C. M., Mach, K. K., & Mayser, P. A. M. (1999). Medium-chain triglycerides inhibit growth of Malassezia: Implications for prevention of systemic infection. Critical Care Medicine.
Puertollano, E., Kolida, S., & Yaqoob, P. (2014). Biological significance of short-chain fatty acid metabolism by the intestinal microbiome. In Current Opinion in Clinical Nutrition and Metabolic Care (Vol. 17, Issue 2, pp. 139-144). https://doi.org/10.1097/MCO.0000000000000025
Putignani, L., Del Chierico, F., Vernocchi, P., Cicala, M., Cucchiara, S., & Dallapiccola, B. (2016). Gut Microbiota Dysbiosis as Risk and Premorbid Factors of IBD and IBS Along the Childhood-Adulthood Transition. In Inflammatory Bowel Diseases (Vol. 22, Issue 2, pp. 487-504). Lippincott Williams and Wilkins. https://doi.org/10.1097/MIB.0000000000000602
Ramakrishna, B. S. (2013). Role of the gut microbiota in human nutrition and metabolism. Journal of Gastroenterology and Hepatology (Australia), 28 (S4), 9-17. https://doi.org/10.1111/jgh.12294.
Rangaraju, V., Lewis, T. L., Hirabayashi, Y., Bergami, M., Motori, E., Cartoni, R., Kwon, S. K., & Courchet, J. (2019). Pleiotropic mitochondria: The influence of mitochondria on neuronal development and disease. Journal of Neuroscience, 39 (42), 8200-8208. https://doi.org/10.1523/JNEUROSCI.1157-19.2019
Ratajczak, W., Rył, A., Mizerski, A., Walczakiewicz, K., Sipak, O., & Laszczyńska, M. (2019). Immunomodulatory potential of gut microbiome-derived shortchain fatty acids (SCFAs). In Acta Biochimica Polonica (Vol. 66, Issue 1, pp. 1-12). Acta Biochimica Polonica. https://doi.org/10.18388/abp.2018_2648
Rauf, A., Khalil, A. A., Rahman, U. ur, Khalid, A., Naz, S., Shariati, M. A., Rebezov, M., Urtecho, E. Z., de Albuquerque, R. D. D. G., Anwar, S., Alamri, A., Saini, R. K., & Rengasamy, K. R. R. (2022). Recent advances in the therapeutic application of short-chain fatty acids (SCFAs): An updated review. In Critical Reviews in Food Science and Nutrition (Vol. 62, Issue 22, pp. 6034-6054). Taylor and Francis Ltd. https://doi.org/10.1080/10408398.2021.1895064
Rebello, C. J., Keller, J. N., Liu, A. G., Johnson, W. D., & Greenway, F. L. (2015). Pilot feasibility and safety study examining the effect of medium chain triglyceride supplementation in subjects with mild cognitive impairment: A randomized controlled trial. BBA Clinical, 3, 123-125. https://doi.org/10.1016/J.BBACLI.2015.01.001
Robles, Aide, Theodora L. Yellowman, Sayalee Joshi, Srivatsan Mohana Rangan, and Anca G. Delgado. 2021. “Microbial chain elongation and subsequent fermentation of elongated carboxylates as H2-producing processes for sustained reductive dechlorination of chlorinated ethenes.” Environmental Science & Technology 55 (15): 10398-10410. doi: 10.1021/acs.est.1c01319.
Roopashree, P. G., Shetty, S. S., & Suchetha Kumari, N. (2021). Effect of medium chain fatty acid in human health and disease. In Journal of Functional Foods (Vol. 87). Elsevier Ltd. https://doi.org/10.1016/j.jff.2021.104724
Schönfeld, P., & Wojtczak, L. (2016). Short-and medium-chain fatty acids in energy metabolism: The cellular perspective. In Journal of Lipid Research (Vol. 57, Issue 6, pp. 943-954). American Society for Biochemistry and Molecular Biology Inc. https://doi.org/10.1194/jlr.R067629
Schulthess, J., Pandey, S., Capitani, M., Rue-Albrecht, K. C., Arnold, I., Franchini, F., Chomka, A., Ilott, N. E., Johnston, D. G. W., Pires, E., McCullagh, J., Sansom, S. N., Arancibia-Cárcamo, C. V., Uhlig, H. H., & Powrie, F. (2019). The Short Chain Fatty Acid Butyrate Imprints an Antimicrobial Program in Macrophages. Immunity, 50 (2), 432-445.e7. https://doi.org/10.1016/j.immuni.2018.12.018
Shilling, M., Matt, L., Rubin, E., Visitacion, M. P., Haller, N. A., Grey, S. F., & Woolverton, C. J. (2013). Antimicrobial effects of virgin coconut oil and its medium-chain fatty acids on clostridium difficile. Journal of Medicinal Food, 16 (12), 1079-1085. https://doi.org/10.1089/jmf.2012.0303
Sklirou, E., Alodaib, A. N., Dobrowolski, S. F., Mohsen, A. W. A., & Vockley, J. (2021). Physiological Perspectives on the Use of Triheptanoin as Anaplerotic Therapy for Long Chain Fatty Acid Oxidation Disorders. Frontiers in Genetics, 11. https://doi.org/10.3389/fgene.2020.598760
Stavoe, A. K. H., & Holzbaur, E. L. F. (2019). Annual Review of Cell and Developmental Biology Autophagy in Neurons. Annu. Rev. Cell Dev. Biol, 35, 477-500. https://doi.org/10.1146/annurev-cellbio-100818
Steinbusch, Kirsten J. J., Hubertus V. M. Hamelers, and Cees J. N. Buisman. 2008. “Alcohol production through volatile fatty acids reduction with hydrogen as electron donor by mixed cultures.” Water research (Oxford) 42 (15): 4059-4066. doi: 10.1016/j.watres.2008.05.032.
Sunkara, L. T., Jiang, W., & Zhang, G. (2012). Modulation of Antimicrobial Host Defense Peptide Gene Expression by Free Fatty Acids. PLoS ONE, 7 (11). https://doi.org/10.1371/journal.pone.0049558
Takikawa, M., Kumagai, A., Hirata, H., Soga, M., Yamashita, Y., Ueda, M., Ashida, H., & Tsuda, T. (2013). 10-Hydroxy-2-decenoic acid, a unique medium-chain fatty acid, activates 5′-AMP-activated protein kinase in L6 myotubes and mice. Molecular Nutrition and Food Research, 57 (10), 1794-1802. https://doi.org/10.1002/mnfr.201300041
Tan, J., Mckenzie, C., Potamitis, M., Thorburn, A. N., Mackay, C. R., & Macia, L. (2014). The Role of Short-Chain Fatty Acids in Health and Disease. Advances in Immunology, 121, 91-119. https://doi.org/10.1016/B978-0-12-800100-4.00003-9
Taylor, M. K., Sullivan, D. K., Mahnken, J. D., Burns, J. M., & Swerdlow, R. H. (2018a). Feasibility and efficacy data from a ketogenic diet intervention in Alzheimer's disease. Alzheimer's & Dementia: Translational Research & Clinical Interventions, 4, 28-36. https://doi.org/10.1016/J.TRCI.2017.11.002.
Thormar, H., & Hilmarsson, H. (2007). The role of microbicidal lipids in host defense against pathogens and their potential as therapeutic agents. Chemistry and Physics of Lipids, 150 (1), 1-11. https://doi.org/10.1016/J.CHEMPHYSLIP.2007.06.220
Tsuji, H., Kasai, M., Takeuchi, H., Nakamura, M., Okazaki, M., & Kondo, K. (2001). Human Nutrition and Metabolism Dietary Medium-Chain Triacylglycerols Suppress Accumulation of Body Fat in a Double-Blind, Controlled Trial in Healthy Men and Women. https://academic.oup.com/jn/article-abstract/131/11/2853/4686757
Tsukuda, N., Yahagi, K., Hara, T., Watanabe, Y., Matsumoto, H., Mori, H., Higashi, K., Tsuji, H., Matsumoto, S., Kurokawa, K., & Matsuki, T. (2021) Key bacterial taxa and metabolic pathways affecting gut short-chain fatty acid profiles in early life, The ISME Journal, 15 (9), 2574-2590. https://doi.org/10.1038/s41396-021-00937-7
Wang, J., Huang, N., Xiong, J., Wei, H., Jiang, S., & Peng, J. (2018). Caprylic acid and nonanoic acid upregulate endogenous host defense peptides to enhance intestinal epithelial immunological barrier function via histone deacetylase inhibition. International Immunopharmacology, 65, 303-311. https://doi.org/10.1016/J.INTIMP.2018.10.022.
Wang, J., Lu, J. X., Xie, X. W., Xiong, J., Huang, N., Wei, H., Jiang, S., & Peng, J. (2018). Blend of organic acids and medium chain fatty acids prevents the inflammatory response and intestinal barrier dysfunction in mice challenged with enterohemorrhagic Escherichia coli O157: H7. International Immunopharmacology, 58, 64-71. https://doi.org/10.1016/J.INTIMP.2018.03.014
Williams, L., Rhodes, K. S., Karmally, W., Welstead, L. A., Alexander, L., & Sutton, L. (2018). Familial chylomicronemia syndrome: Bringing to life dietary recommendations throughout the life span. Journal of Clinical Lipidology, 12 (4), 908-919. https://doi.org/10.1016/J.JACL.2018.04.010
Wlaź, P., Socała, K., Nieoczym, D., Łuszczki, J. J., Arnowska, I., Arnowski, T., Czuczwar, S. J., & Gasior, M. (2012). Anticonvulsant profile of caprylic acid, a main constituent of the medium-chain triglyceride (MCT) ketogenic diet, in mice. Neuropharmacology, 62 (4), 1882-1889. https://doi.org/10.1016/J.NEUROPHARM.2011.12.015
Wlaź, P., Socała, K., Nieoczym, D., Zarnowski, T., Zarnowska, I., Czuczwar, S. J., & Gasior, M. (2015). Acute anticonvulsant effects of capric acid in seizure tests in mice. Progress in Neuro-Psychopharmacology and Biological Psychiatry, 57, 110-116. https://doi.org/10.1016/J.PNPBP.2014.10.013
Xiong, R. G., Zhou, D. D., Wu, S. X., Huang, S. Y., Saimaiti, A., Yang, Z. J., Shang, A., Zhao, C. N., Gan, R. Y., & Li, H. Bin. (2022). Health Benefits and Side Effects of Short-Chain Fatty Acids. In Foods (Vol. 11, Issue 18). MDPI. https://doi.org/10.3390/foods11182863
Xu, Q., Zhang, Y., Zhang, X., Liu, L., Zhou, B., Mo, R., Li, Y., Li, H., Li, F., Tao, Y., Liu, Y., & Xue, C. (2020). Medium-chain triglycerides improved cognition and lipid metabolomics in mild to moderate Alzheimer's disease patients with APOE4−/−: A double-blind, randomized, placebo-controlled crossover trial. Clinical Nutrition, 39 (7), 2092-2105. https://doi.org/10.1016/J.CLNU.2019.10.017
Yan, D., Ye, S., He, Y., Wang, S., Xiao, Y., Xiang, X., Deng, M., Luo, W., Chen, X., & Wang, X. (2023). Fatty acids and lipid mediators in inflammatory bowel disease: from mechanism to treatment. In Frontiers in Immunology (Vol. 14). Frontiers Media SA. https://doi.org/10.3389/fimmu.2023.1286667
Yin, Yanan, Yifeng Zhang, Dimitar Borisov Karakashev, Jianlong Wang, and Irini Angelidaki. 2017. “Biological caproate production by Clostridium kluyveri from ethanol and acetate as carbon sources.” Bioresource Technology 241:638-644. doi: 10.1016/j.biortech.2017.05.184
Zakaria, Z. A., Rofiee, M. S., Somchit, M. N., Zuraini, A., Sulaiman, M. R., Teh, L. K., Salleh, M. Z., & Long, K. (2011). Hepatoprotective activity of dried-and fermented-processed virgin coconut oil. Evidence-Based Complementary and Alternative Medicine, 2011. https://doi.org/10.1155/2011/142739
Zeng, Q., Stylianou, T., Preston, J., Glover, S., O'neill, K., Woolf, E. C., Scheck, A. C., & Syed, N. (n.d.). The ketogenic diet alters the epigenetic landscape of GBM to potentiate the effects of chemotherapy and radiotherapy. https://academic.oup.com/neuro-oncology/article/21/Supplement_4/iv8/5586392
Zeng, X., Sunkara, L. T., Jiang, W., Bible, M., Carter, S., Ma, X., Qiao, S., & Zhang, G. (2013). Induction of Porcine Host Defense Peptide Gene Expression by Short-Chain Fatty Acids and Their Analogs. PLoS ONE, 8 (8). https://doi.org/10.1371/journal.pone.0072922
Zentek, J., Buchheit-Renko, S., Männer, K., Pieper, R., & Vahjen, W. (2012). Intestinal concentrations of free and encapsulated dietary medium-chain fatty acids and effects on gastric microbial ecology and bacterial metabolic products in the digestive tract of piglets. Archives of Animal Nutrition, 66 (1), 14-26. https://doi.org/10.1080/1745039X.2011.644916

We claim:

1. A composition comprising a substrate for chain elongation by gut microbiome, wherein:
the substrate for chain elongation is selected from ethanol, lactate, or a combination thereof; and
the composition is formulated for colonic delivery.

2. The composition of claim 1, wherein composition further comprises a colonic delivery system that encapsulates the at least one substrate for chain elongation.

3. The composition of claim 2, wherein the colonic delivery system comprises a plurality of polymers, wherein at least one polymer of the plurality of polymers prevent penetration of gastric fluid into the colonic delivery system and the plurality of polymers further comprises a pH-independent erodible polymer that delays the release of the at least one substrate for chain elongation for at least four hours after gastric emptying.

4. The composition of claim 3, wherein the colonic delivery system further comprises a metal compound, wherein the metal compound is incorporated into at least one of the plurality of polymers.

5. The composition of claim 2, wherein the colonic delivery system comprises a suppository base coated with a hydrophobic polymer, wherein the composition is a capsule and the substrate for chain elongation is dispersed in the suppository base.

6. The composition of claim 2, wherein the colonic delivery system comprises a food component selected from the group consisting of: a non-starch polysaccharide, amylose, xanthan gum, and an azopolymer, wherein the colonic delivery system is specifically decomposed by an enzyme specific to the colon compared to other sites in the gastrointestinal tract.

7. The composition of claim 2, wherein the colonic delivery system comprises:
a liquid, semi-solid, or solid core, wherein the liquid, semi-solid, or solid core contains the substrate for chain elongation; and
an outer shell layer, wherein the outer shell layer encapsulates the liquid, semi-solid, or solid core and the outer shell layer ensures colonic delivery of the substrate for chain elongation.

8. The composition of claim 7, wherein the colonic delivery system comprises:
a liquid, semi-solid, or solid core, wherein the liquid, semi-solid, or solid core contains the substrate for chain elongation;
an outer shell layer, wherein the outer shell layer encapsulates the liquid, semi-solid, or solid core; and
a modified release coating, wherein the modified release coating is applied to the outer shell layer and ensures colonic delivery of the substrate for chain elongation.

9. The composition of claim 2, wherein the concentration of the substrate for chain elongation in the composition is at least 1 mM.

10. The composition of claim 2, wherein the concentration of the substrate for chain elongation in the composition is 1 mM-120 mM.

11. The composition of claim 2, wherein the substrate for chain elongation is ethanol, the concentration of ethanol in the composition is 4 mM-100 mM.

12. The composition of claim 9, wherein the concentration of ethanol in the composition is 40 mM-100 mM.

13. The composition of claim 6, wherein the concentration of ethanol in the composition is 4 mM-40 mM.

14. The composition of claim 2, wherein the substrate for chain elongation is lactate, the concentration of lactate in the composition is 1 mM-120 mM.

15. The composition of claim 14, wherein concentration of lactate in the composition is 1 mM-100 mM.

16. The composition of claim 14, wherein concentration of lactate in the composition is 1 mM-40 mM.

17. The composition of claim 12, wherein concentration of lactate in the composition is 4 mM-40 mM.

18. The composition of claim 1, further comprising acetate or butyrate.

19. A method of increasing production of medium-chain and/or small chain fatty acids in the digestive tract of a subject, the method comprising administering to the subject the composition of claim 1.

20. A method of ameliorating the symptoms of diabetes, obesity, Alzheimer's disease, Parkinson's disease, Amyotrophic Lateral Sclerosis (ALS), inflammatory bowel diseases (ulcerative colitis, UC, and Crohn's disease, CD), cancer (particularly colon cancer), and/or cardiomyopathies comprising administering to the subject the composition of claim 1.
